SEQUENCE LISTING (1 sequence)

SEQ ID NO: 1 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

(19) United States
(12) Patent Application Publication
(10) Pub. No.: US 2026/0104419 A1
(43) Pub. Date: Apr. 16, 2026

(54) METHODS FOR IDENTIFYING SUBJECTS AT RISK OF THROMBOSIS OR MALIGNANCY

(71) Applicant: The Johns Hopkins University, Baltimore, MD (US)

(72) Inventors: Eduardo Gómez-Bañuelos, Baltimore, MD (US); Felipe Andrade, Baltimore, MD (US)

(21) Appl. No.: 19/355,281

(22) Filed: Oct. 10, 2025

Related U.S. Application Data
(60) Provisional application No. 63/706,148, filed on Oct. 11, 2024.

(51) Int. Cl. G01N 33/574; G01N 33/543

(52) U.S. Cl. CPC G01N 33/57585; G01N 33/543; G01N 2333/4703; G01N 2470/04

(57) ABSTRACT

Disclosed herein are methods for detecting or determining an amount, quantity, concentration and/or level of an anti-transcription factor A, mitochondrial (TFAM) antibody, such as an anti-human TFAM antibody, in one or more biologics samples obtained from a subject. In some aspects, the methods relate to identifying a subject suffering from systemic lupus erythematosus (SLE) and/or antiphospholipid syndrome that is at risk of thrombosis, malignancy, death or any combination thereof.

### RELATED APPLICATION INFORMATION

This application claims priority to U.S. Application No. 63/706,148 filed on Oct. 11, 2024, the contents of which are herein incorporated by reference.

### STATEMENT REGARDING FEDERALLY SPONSORED RESEARCH

This invention was made with government support under NIH/NIAID R21AI169851 awarded by the National Institutes of Health. The government has certain rights in the invention.

### TECHNICAL FIELD

The present disclosure relates to methods for identifying the presence of or detecting or determining an amount, quantity, concentration and/or level of an anti-transcription factor A, mitochondrial (TFAM) antibody, such as an anti-human TFAM antibody, in one or more biological samples obtained from a subject. In some aspects, the methods relate to identifying a subject suffering from systemic lupus erythematosus (SLE) and/or antiphospholipid syndrome that is at risk of thrombosis, malignancy and/or death.

### SEQUENCE LISTING STATEMENT

The contents of the electronic sequence listing titled JHU_43678_202_SequenceListing.xml (Size: 2,171 bytes; and Date of Creation: Oct. 10, 2025) are herein incorporated by reference in their entirety.

### BACKGROUND

Systemic lupus erythematosus (SLE) is an autoimmune disease in which the immune system erroneously attacks healthy tissues, resulting in inflammation and damage to various organs, including the skin, joints, kidneys, and brain. The global prevalence of SLE is estimated to range from 20 to 150 cases per 100,000 people, affecting approximately 5 million individuals worldwide. However, these statistics can vary significantly due to several factors, including underdiagnosis, variations in clinical presentation, and disparities in healthcare access. Furthermore, patients with SLE face an increased risk of developing thrombosis (blood clots) which is a critical concern because thrombosis can lead to severe complications, such as strokes, heart attacks, and pulmonary embolism, significantly impacting patients' overall health and quality of life. Currently, the evaluation of SLE involves a combination of clinical assessments, laboratory tests, and imaging studies to monitor symptoms and organ involvement. However, these methods face significant challenges. The variability of symptoms can make diagnosis and assessment difficult, as they may be nonspecific and fluctuate over time. Additionally, there is a lack of standardized criteria for diagnosing SLE, leading to reliance on clinical judgment and subjective assessments. This reliance can result in misdiagnosis or delayed diagnosis, as the symptoms of SLE often vary widely among patients and may mimic other conditions. Consequently, distinguishing between SLE and related complications, such as antiphospholipid syndrome (APS), becomes particularly challenging. Thus, there is an urgent need for distinct clinical and transcriptional subsets of the disease that are associated with key complications in SLE. Moreover, existing evaluation methods do not adequately assess the risk of complications such as thrombosis. These issues can result in delays in diagnosis, inappropriate management, and an increased risk of complications for patients with SLE. Therefore, establishing standardized approaches to identify SLE patients at risk of thrombosis, independent of traditional APS-associated antibodies, is essential.

### SUMMARY

The present disclosure relates to methods for detecting or determining an amount, quantity, concentration and/or level of an antibody (anti-TFAM antibody) in one or more samples obtained from a subject. In some aspects, the methods relate to identifying a subject suffering from systemic lupus erythematosus (SLE) that is at risk of thrombosis or malignancy.

The present disclosure relates to a method of identifying a subject suffering from systemic lupus erythematosus (SLE) that is at risk of thrombosis or malignancy, the method comprising the steps of:
(a.) contacting at least one biological sample obtained from a subject suffering from SLE with:
(i.) at least one first specific binding partner, wherein the at least one first specific binding partner comprises a polypeptide comprising or consisting of amino acids 43 to 246 of mature human transcription factor A, mitochondrial (TFAM), which is also referred to herein as a capture reagent or capture polypeptide, and further wherein the at least one anti-TFAM antibody in the sample specifically binds to the polypeptide, and
(ii.) at least one type of second specific binding partner comprising a detectable label, which is referred to herein as a detection reagent, wherein the second specific binding partner specifically binds to at least one anti-TFAM antibody in the sample.
thereby producing one or more types of first complexes comprising the first specific binding partner-anti-TFAM antibody-second specific binding partner;
(b.) assessing a signal from one or more types of first complexes, wherein the amount of detectable signal from the detectable label indicates the amount of at least one anti-TFAM antibody in the sample; and
(c.) identifying the subject as at risk of thrombosis or for malignancy if the amount of first complexes in the sample higher than a reference level or not at risk of thrombosis or for malignancy if the amount of first complexes in the sample is equal to or lower than a reference level, wherein the reference level is based on the amount of first complexes detected in a control group of human subjects who do not suffer from SLE.

In some aspects of the above method, the human TFAM has at least 80%, at least 85%, at least 90%, at least 95%, at least 99% or at least 100% sequence identity to SEQ ID NO:1. In some aspects of the above method, the human TFAM has at least 80% sequence identity to SEQ ID NO: 1. In some aspects of the above method, the human TFAM has at least 85% sequence identity to SEQ ID NO:1. In some aspects of the above method, the human TFAM has at least 90% sequence identity to SEQ ID NO:1. In some aspects of the above method, the human TFAM has at least 95% sequence identity to SEQ ID NO:1. In some aspects of the above method, the human TFAM has at least 99% sequence identity to SEQ ID NO: 1. In some aspects of the above method, the human TFAM has at least 100% sequence identity to SEQ ID NO:1.

In some aspects of the above method, the first specific binding partner binds to a first epitope on an anti-TFAM antibody and the second specific binding partner binds to an antigen binding site or paratope (e.g., a second epitope) on the anti-TFAM antibody.

In some aspects of the above method, the biological sample is a body fluid from a human subject.

In some aspects of the above method, the body fluid is selected from the group consisting of whole blood, plasma, serum, salvia, ascites fluid and bronchoalveolar lavage. In some aspects of the above method, the body fluid is whole blood. In some aspects of the above method, the body fluid is plasma. In some aspects of the above method, the body fluid is serum. In some aspects of the above method, the body fluid is salvia. In some aspects of the above method, the body fluid is ascites fluid. In some aspects of the above method, the body fluid is bronchoalveolar lavage.

In some aspects of the above method, the at least one first specific binding partner or the at least one second specific binding partner is immobilized on a solid support.

In some aspects of the above method, the second specific binding partner comprises a anti-human IgG antibody. In some aspects of the above method, the second specific binding partner comprises a anti-human IgA antibody. In some aspects of the above method, the second specific binding partner comprises a anti-human IgD antibody. In some aspects of the above method, the second specific binding partner comprises a anti-human IgE antibody. In some aspects of the above method, the second specific binding partner comprises a anti-human IgM antibody.

In some aspects of the above method, the reference level is at least one standard deviation above a mean amount of first complexes detected in a control group of human subjects who do not suffer from SLE.

In some aspects of the above method, the reference level is at least two standard deviations above a mean amount of first complexes detected in a control group of human subjects who do not suffer from SLE.

In further aspects, the above method is selected from the group consisting of: an immunoassay, a clinical chemistry assay, and a lateral flow assay. In further aspects, the above method is selected from the group consisting of: an immunoassay. In further aspects, the above method is selected from the group consisting of: a clinical chemistry assay. In further aspects, the above method is selected from the group consisting of: a lateral flow assay.

In still further aspects, the above method is adapted for use in an automated system or a semi-automated system. In still further aspects, the above method is adapted for use in an automated system. In still further aspects, the above method is adapted for use in a semi-automated system.

In some aspects, the above method, the subject is identified as at risk of new or recurrent thrombotic events. In some aspects, the above method, the subject is identified as at risk of new thrombotic events. In some aspects, the above method, the subject is identified as at risk of recurrent thrombotic events.

In yet further aspects, the above method comprises treating the subject identified as at risk of new or recurrent thrombotic events with at least one antithrombotic agents. In yet further aspects, the above method comprises treating the subject identified as at risk of new thrombotic events with at least one antithrombotic agents.

In yet further aspects, the above method comprises treating the subject identified as at risk of recurrent thrombotic events with at least one antithrombotic agents.

In some aspects, the antithrombotic agents is low-dose aspirin, direct factor Xa inhibitors, thrombin inhibitors, PY12 inhibitors, low molecular weight inhibitors, warfarin, heparin, or a combination thereof. In some aspects, the antithrombotic agents is low-dose. In some aspects, the antithrombotic agents is direct factor Xa inhibitors. In some aspects, the antithrombotic agents is thrombin inhibitors. In some aspects, the antithrombotic agents is PY12 inhibitors. In some aspects, the antithrombotic agents is low molecular weight inhibitors. In some aspects, the antithrombotic agents is warfarin. In some aspects, the antithrombotic agents is heparin.

In some aspects, the subject is identified as at risk for malignancy.

In some aspects, the subject is monitored for developing a malignancy.

### BRIEF DESCRIPTION OF THE FIGURES

The patent or application file contains drawings executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIGS. 1A-1G show that TFAM is a novel autoantigen in SLE. FIG. 1A shows healthy control neutrophils were stained with SLE serum detecting cytoplasmic antigens (green), anti-TFAM monoclonal antibody C9 (red), and DAPI (blue) and viewed by immunofluorescence microscopy. Individual and merged images are shown. FIG. 1B shows TFAM structure predicted by AlphaFold. Amino acids (aa) 1-42 form the mitochondrial targeting sequence (MTS), while aa 43-246 comprise the mature form of TFAM, which includes 2 high-mobility group domains (HMG1 and HMG2), a linker, and a C-terminal tail (C-tail). FIG. 1C shows the serum levels of anti-TFAM antibodies in SLE (n=22) and healthy controls (n=10). The P value was obtained using the Wilcoxon rank sum test. FIG. 1D shows representative immunoblots showing the reactivity of healthy control serum (n=4) and anti-TFAM-positive SLE serum (n=4) against human recombinant TFAM. FIG. 1E shows the serum levels of anti-TFAM antibodies before and after blocking with human recombinant HMG box 1 (HMGB1) protein (n=9). Comparisons were done using a paired samples Wilcoxon signed-rank test. FIG. 1F shows HEp-2 cells were stained using anti-TFAM-positive SLE serum (green) and anti-TFAM monoclonal antibody C9 (red) and viewed by immunofluorescence microscopy. Individual and merged images are shown. FIG. 1F shows HEp-2 cells were stained with anti-TFAM-positive SLE serum (green) in the absence (left) and presence (right) of blocking with recombinant TFAM (rTFAM). Representative images of 12 (FIG. 1F) and 8 (FIG. 1G) anti-TFAM-positive SLE sera are shown. FIG. 1G shows anti-TFAM-positive SLE sera. DAPI, 4′,6-diamidino-2-phenylindole; SLE, systemic lupus erythematosus; TFAM, transcription factor A, mitochondrial.

FIGS. 2A-2E show anti-TFAM antibodies are prevalent in SLE. FIG. 2A shows serum levels of anti-TFAM antibodies in SLE patients (n=158) from the SPARE lupus cohort compared with those of healthy controls (HC, n=98), primary antiphospholipid syndrome (PAPS, n=50), rheumatoid arthritis (RA, n=36), and dermatomyositis (DM, n=40). Comparisons between groups were performed using one-way analysis of variance and Tukey's post hoc test. FIG. 2B shows the frequency of anti-TFAM antibodies in each group in FIG. 2A. FIG. 2C shows P values indicating significant differences between the frequency in FIG. 2B of anti-TFAM in each group from FIG. 2A. P values were obtained using Fisher's exact test. FIG. 2D shows the association between anti-dsDNA positivity and anti-TFAM antibodies. The association was estimated using Fisher's exact test. FIG. 2E shows the SLEDAI score at the time of visit in SLE patients according to anti-TFAM positivity. The P value was obtained using a Student's t test. AU, arbitrary units; dsDNA, double-stranded DNA; SLE, systemic lupus erythematosus; SLEDAI, Systemic Lupus Erythematosus Disease Activity Index: SPARE, Study of biological Pathways, Disease Activity and Response markers in patients with Systemic Lupus Erythematosus; TFAM, transcription factor A, mitochondrial.

FIG. 3A and FIG. 3B show that anti-TFAM antibodies are associated with APS and thrombotic events in SLE. Clinical and immunological characteristics of SLE patients according to anti-TFAM (FIG. 3A) and anti-dsDNA antibody positivity (FIG. 3B). The graphs represent data for 157 SLE patients, including 48 positive and 109 negatives for anti-TFAM antibodies. Each dot represents a clinical manifestation. Only clinical manifestations significantly associated (P<0.05) with anti-TFAM and anti-dsDNA antibodies were labeled. Quantities under each label correspond to the odds ratio (OR) and 95% confidence interval (CI) of anti-TFAM (+) vs. anti-TFAM (−) (FIG. 3A) and anti-dsDNA (+) vs. anti-dsDNA (−) (FIG. 3B) calculated using a 2×2 table. P values were obtained by Fisher's exact test. ACL, anti-cardiolipin antibodies; APS, antiphospholipid syndrome; AT, any thrombotic event; B2GPI, anti-β2-glycoprotein-I antibodies; CH50, 50% hemolytic complement test; CV, cardiovascular; dsDNA, anti-double-stranded DNA antibodies; DVT, deep vein thrombosis; ESR, erythrocyte sedimentation rate; FPRPR, false positive rapid plasma reagin test; GI, gastrointestinal; LAC, lupus anticoagulant; MS, musculoskeletal; P, pulmonary; Renal insuff, renal insufficiency; Ro52, anti-Ro52 antibodies; S, Serositis; SLE, systemic lupus erythematosus; Sm, anti-Sm antibodies; TFAM, transcription factor A, mitochondrial; VT, venous thrombosis.

FIGS. 4A-4E show that anti-TFAM antibodies are predictive of thrombotic events in SLE. FIG. 4A shows univariate logistic regression showing the predictive value of anti-TFAM antibodies and significantly associated factors from Table 3 for any thrombotic events in patients with SLE. FIGS. 4B and 4C show multivariate logistic regression models analyzing the predictive value of anti-TFAM antibodies after adjustment for disease duration, lupus anticoagulant (LAC), smoking, alcohol use, disability, and photosensitivity. FIGS. 4D-4E show logistic regression models showing the additive value of a history of smoking or LAC with anti-TFAM antibodies in SLE. The graphs represent data for 157 SLE patients, including 48 positive and 109 negatives for anti-TFAM antibodies. AUC, area under the curve; LAC, lupus anticoagulant; OR, odds ratio; SLE systemic lupus erythematosus; TFAM, transcription factor A, mitochondrial.

FIGS. 5A-5H show that anti-TFAM antibodies are linked to a thrombotic transcriptome in SLE. FIG. 5A shows principal component (PC) analysis of the top 500 transcripts ranked by their Fisher score between in anti-TFAM positive vs. anti-TFAM negative SLE. FIG. 5B shows PC1 levels in patients with SLE according to the presence of both anti-TFAM antibodies and a history of any thrombotic event (AT). Comparisons were done using ANOVA and Tukey's test as post-hoc. FIGS. 5C-5D show a correlation between Anti-TFAM PC1 and anti-dsDNA PC1 (FIG. 5C) and anti-TFAM PC1 and thrombosis PC1 (FIG. 5D). Correlations were estimated with Pearson's r correlation coefficient. FIG. 5E shows enrichment analysis of significantly correlated transcripts with anti-TFAM, thrombosis, and anti-dsDNA PC1. Significantly, correlated genes were determined by Pearson's r correlation coefficients. Only, genes with an adjusted p value <0.01 (Benjamini-Hochberg's method) were considered in the analyses. The enrichment analysis was conducted using the Metanalysis function from Metascape.org. For this data set, a total of 156 microarrays were analyzed, of which 47 and 109 correspond to anti-TFAM positive and anti-TFAM negative SLE. FIGS. 5F-5H show the activity levels of IFN-I, IFN-II and IFN-III in patients with SLE according to anti-TFAM positivity. Anti-TFAM positive, n=47; anti-TFAM negative, n=109. A comparison of IFN levels was done with Student's T test.

FIG. 6 shows that SLE autoantibodies target nuclear, nucleolar and cytoplasmic antigens in neutrophils. Healthy control neutrophils were fixed, permeabilized and stained with SLE serum and Alexa Fluor 488 goat anti-human IgG (green), as well as DAPI (blue), and examined by immunofluorescence microscopy. Individual and merged images are shown. Representative neutrophil staining patterns detected by SLE sera (n=15) are shown.

FIGS. 7A and 7B show anti-TFAM antibodies are stable over time and are not cross reactive with β2GPI. FIG. 7A shows serum levels of anti-TFAM antibodies were measured in 18 SLE patients in two time points with a median (min, max) interval of 334 (91, 742) days after their first anti-TFAM positive test. Comparisons were done using a paired samples t-test. FIG. 7B shows serum levels of anti-TFAM antibodies were detected by ELISA before and after blocking with β2GPI. Comparisons were done using a paired samples Wilcoxon signed-rank test.

FIGS. 8A-8C show anti-TFAM antibodies are associated with increased damage accrual, malignancy risk and mortality in SLE. FIG. 8A shows the SLICC score according to anti-TFAM positivity. Anti-TFAM positive, n=47. Anti-TFAM negative, n=110. Comparison of SLICC scores was done using Student's T test. Malignancy (FIG. 8B) and death (FIG. 8C) OR in SLE patients according to autoantibody positivity.

### DETAILED DESCRIPTION

The present disclosure relates to methods for detecting the presence of or determining an amount, quantity, concentration and/or level of an antibody (anti-TFAM antibody) in one or more samples obtained from a subject. In some aspects, the methods relate to identifying a subject that is at risk of thrombosis, malignancy, death, or any combination thereof. In some aspects, the subject is suffering from systemic lupus erythematosus, antiphospholipid syndrome, a combination thereof.

### Definitions

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

The terms “comprise(s),” “include(s),” “having.” “has.” “can,” “contain(s),” and variants thereof, as used herein, are intended to be open-ended transitional phrases, terms, or words that do not preclude the possibility of additional acts or structures. The singular forms “a.” “and” and “the” include plural references, i.e., “one or more.” unless the context clearly dictates otherwise.

The present disclosure also contemplates other embodiments “comprising.” “consisting of” and “consisting essentially of,” the embodiments or elements presented herein, whether explicitly set forth or not.

The term “about,” when used in connection with one or more numbers or numerical ranges, should be understood to refer to all such numbers, including all numbers in a range and modifies that range by extending the boundaries slightly above and slightly below the numerical values set forth by, for example, in some embodiments, +/−20%, +/−15%, +/−10%, +/−5%, +/−4%, +/−3%, +/−2%, and +/−1%. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The terms “antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and WO 2001/058956, the contents of each of which are herein incorporated by reference), or domain antibodies (dAbs) (e.g., such as described in Holt et al., Trends in Biotechnology, 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody”.

The term “antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The term “anti-species antibodies” as used herein refers to an antibody, such as an IgG, IgE, IgM, IgD, IgA, and/or IgY antibody, that recognize antibodies of another species of interest, such as, for example, a goat, rabbit, mouse, sheep, donkey, chicken, primate, or human. For example, in some aspects, the anti-species antibodies are anti-human antibodies, e.g., anti-human IgA, IgD, IgE and/or IgM antibodies, that recognize other human IgA, IgD, IgE, IgG, and/or IgM, antibodies.

The term “autoantibodies” refers to antibodies that are capable of reacting against an antigenic constituent of an individual's own tissue or cells (e.g., the antibodies recognize and bind to “self-antigens” or autoantigens).

The term “autoantigens” or “self-antigens” as used interchangeably herein, refers to specific proteins, glycoproteins, or other biomolecules derived from a subject's own tissues or cells that elicit an immune response, resulting in the activation of autoreactive T cells and the production of autoantibodies. These self-antigens may include proteins found in the nucleus, cytoplasm, or cell membrane of cells and are implicated in various autoimmune diseases, where the immune system erroneously targets them, leading to inflammation and tissue damage.

The terms “component.” “components.” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, an anal swab specimen, a nasal mucus specimen, serum or plasma sample, an oropharyngeal specimen or a nasopharyngeal specimen, in accordance with the methods described herein and other methods known in the art. Some components can be in solution or lyophilized for reconstitution for use in an assay

The term “controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

The term “damage-associated molecular patterns” (DAMPs) refers to endogenous molecules released by stressed or damaged cells that act as signals to alert the immune system to tissue injury. These molecules can include proteins, nucleic acids, and other cellular components that, when released into the extracellular environment, trigger inflammatory responses and activate innate immune pathways.

The terms “epitope,” or “epitopes.” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

The term “humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2. CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art. The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences.

The term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (V erlagsgesellschaft, Weinheim, 1987)).

The term “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

The terms “identical” or “identity.” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, di viding the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

The terms “label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, introduction to Immunocytochemistry, 2nd ed., Springer Verlag, N.Y. (1997), and in Haugland, Handbook of Fluorescent Probes and Research Chemicals, (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett., 16: 1324-1328 (2006); Adamczyk et al., Bioorg. Med. Chem. Lett., 4: 2313-2317 (2004); Adamczyk et al., Biorg. Med. Chem. Lett., 14: 3917-3921 (2004); and Adamczyk et al., Org. Lett., 5: 3779-3782 (2003)).

As used herein, the term “microparticle(s)” refers to small particles with dimensions typically ranging from 0.1 to 100 micrometers (μm). These particles can be solid or colloidal in nature. Microparticles can include, but are not limited to, polymeric microparticles, liposomes, microspheres, nanoparticles, magnetic microparticles, non-magnetic microparticles, protein microparticles, biodegradable microparticles, ceramic microparticles, hollow microparticles, Janus particles, nanospheres, microcapsules, and nanocapsules. In some cases, microparticle can include one or more of the following: a poly (lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids, in any combination thereof.

The term “non-point-of-care device” refers to a device that is not a point-of-care device or a single use device. A non-point-of-care device refers to any device that does not meet any of the above limitations of a point-of-care or a single use device as defined herein. In some embodiments, the non-point-of-care device may be a relatively large instrument, such as a tabletop instrument. Accordingly, in some embodiments the non-point-of-care device is not a handheld instrument. In some embodiments, the non-point-of-care device is capable of performing an assay on more than one clinical sample simultaneously.

The term “point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). In some embodiments, the point-of-care device is a single-use device. The term “single-use device” or “single-use instrument” refers to a clinical diagnostic instrument that processes and performs a clinical diagnostic assay on a unit use basis (such as, for example, a single-use cartridge) for a single patient sample. A point-of-care instrument does not perform an assay on more than one clinical sample simultaneously. However, the point-of-care instrument may have the capability to measure more than one parameter (e.g., more than one analyte) in an individual clinical sample per unit use basis.

The terms “recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described herein (Dual-variable domain immunoglobulins and methods for making them are described in Wu, C., et al., Nature Biotechnology, 25: 1290-1297 (2007)). The term “bifunctional antibody.” as used herein, refers to an antibody that comprises a first arm having a specificity for one antigenic site and a second arm having a specificity for a different antigenic site, i.e., the bifunctional antibodies have a dual specificity.

The term “reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

This disclosure provides exemplary reference levels. However, it is well-known that reference levels may vary depending on the nature of the immunoassay (e.g., capture and detection reagents employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

The terms “sample.” “test sample.” “specimen,” “sample from a subject,” “biological sample,” and “patient sample” as used interchangeably herein may be a sample of blood, such as whole blood (including for example, capillary blood, venous blood, dried blood spot, etc.), tissue, urine, saliva, nasal mucus, serum, plasma, amniotic fluid, lower respiratory specimens such as, but not limited to, sputum, endotracheal aspirate or bronchoalveolar lavage, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art Additionally, the sample can be a nasopharyngeal or oropharyngeal sample obtained using one or more swabs that, once obtained, is placed in a sterile tube containing a virus transport media (VTM) or universal transport media (UTM), for testing. Moreover, the sample can be a nasal mucus specimen.

A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, an anal swab specimen, frozen sections taken for histologic purposes, blood (such as whole blood, dried blood spots, etc.), plasma, serum, red blood cells, platelets, interstitial fluid, cerebrospinal fluid, etc. Cell types and tissues may also include lymph fluid, cerebrospinal fluid, or any fluid collected by aspiration. A. tissue or cell type may be provided by removing a sample of cells from a human and a non-human animal but can also be accomplished by using previously isolated cells (e.g., isolated by another person, at another time, and/or for another purpose). Archival tissues, such as those having treatment or outcome history, may also be used. Protein or nucleotide isolation and/or purification may not be necessary. In some embodiments, the sample is a whole blood sample.

In some embodiments, the sample is a capillary blood sample. In some embodiments, the sample is a dried blood spot. In some embodiments, the sample is a serum sample. In yet other embodiments, the sample is a plasma sample. In some embodiments, the sample is an oropharyngeal specimen. In other embodiments, the sample is a nasopharyngeal specimen. In other embodiments, the sample is sputum. In other embodiments, the sample is endotracheal aspirate. In still yet other embodiments, the sample is bronchoalveolar lavage. In still other aspects, the sample can be a nasal mucus specimen. In still other aspects, the sample can be an anal swab specimen.

The term “sensitivity” of an assay as used herein refers to the proportion of subjects for whom the outcome is positive that are correctly identified as positive (e.g., correctly identifying those subjects with a disease or medical condition for which they are being tested).

The term “specificity” of an assay as used herein refers to the proportion of subjects for whom the outcome is negative that are correctly identified as negative (e.g., correctly identifying those subjects who do not have a disease or medical condition for which they are being tested).

The terms “solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner, or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

The terms “specific binding”, “specifically binding”, or “specifically binds” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The terms “specific binding partner” or “specific binding member”, as used interchangeable herein, is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

The term “statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

The terms “subject” and “patient” as used herein interchangeably refers to any vertebrate, including, but not limited to, a mammal (e.g., a bear, cow, cattle, chicken, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgous or rhesus monkey, chimpanzee, etc.) and a human). In some embodiments, the subject may be a human, a non-human primate or a cat. In some embodiments, the subject is a human. In some embodiments, the subject is suffering from SLE. In other embodiments, the subject is suffering from antiphospholipid syndrome. In still other embodiments, the subject is suffering from SLE and antiphospholipid syndrome. In yet still further embodiments, the subject or patient may be undergoing treatment. In yet still further embodiments, the subject or patient undergoing treatment is suffering from SLE and/or antiphospholipid syndrome.

The term “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

The terms “thrombosis” or “thrombotic event” as used interchangeably herein refers to when a blood clot forms in at least one vein, at least one artery, and/or in at least one chamber of the heart of a subject. Diseases caused by thrombosis include stroke, heart attack, peripheral vascular disease, superficial venous thrombosis, deep vein thrombosis (DVT) and pulmonary embolism.

The terms “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The term “variant” is used herein to describe a peptide or polypeptide that differs from a reference peptide or polypeptide in amino acid sequence by the insertion, deletion, or conservative substitution of amino acids, but retains at least one biological activity. Representative examples of “biological activity” include the ability to be bound by a specific antigen or antibody, or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol., 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically-reactive fragment of an anti-analyte antibody that differs from the corresponding fragment of anti-analyte antibody in amino acid sequence but is still antigenically reactive and can compete with the corresponding fragment of anti-analyte antibody for binding with the analyte. “Variant” also can be used to describe a polypeptide or a fragment thereof that has been differentially processed, such as by proteolysis, phosphorylation, or other post-translational modification, yet retains its antigen reactivity.

The term “vector” is used herein to describe a nucleic acid molecule that can transport another nucleic acid to which it has been linked. One type of vector is a “plasmid”, which refers to a circular double-stranded DNA loop into which additional DNA segments may be ligated.

Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

The term “macrophages” refers to a type of immune cell derived from monocytes that play a vital role in the body's immune response. These cells are found throughout the body in various tissues and are key players in both innate and adaptive immunity.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

2. Methods for Detecting the Presence or Determining an Amount, Quantity, Concentration and/or Level of at Least One Anti-TFAM Antibody in One or More Biological Samples Obtained from a Subject

The present disclosure relates to methods or assays for (a) detecting the presence of at least one type of anti-TFAM antibody (e.g., such as at least one anti-human TFAM antibody); or (b) determining or measuring the quantity, amount, level or concentration of at least one type of anti-TFAM antibody (e.g., such as at least one anti-human TFAM antibody), in one or more biological samples obtained from one or more subjects. In some aspects, the present disclosure relates to methods or assays for determining the immune status of a subject. In some aspects, the immune status of a subject is for a subject suffering from systemic lupus erythematosus (SLE) and/or antiphospholipid syndrome. In other aspects, the immune status is determined for a subject that is not suffering from SLE and/or antiphospholipid syndrome. In other aspects, the methods or assays described herein can be used as an aid in determining whether a subject is at risk of thrombosis, malignancy or cancer, and/or death. In some aspects, the subject is suffering from SLE. In some aspects, the subject is suffering from antiphospholipid syndrome. In yet other aspects, the subject is suffering from SLE and antiphospholipid syndrome. In still yet other aspects, the subject is not suffering from SLE and/or antiphospholipid syndrome. In yet further aspects, when determining whether a subject is at risk of thrombosis, the subject may have never previously experienced a thrombotic event and thus is at risk of experiencing a “new” thrombotic event. In other aspects, when determining whether a subject is at risk of thrombosis, the subject may have previously experienced at least one thrombotic event and thus is at risk of experiencing a “recurrent” thrombotic event. In other aspects, when determining whether a subject is at risk of thrombosis, the subject may have never previously experienced a thrombotic event but is suffering from SLE. In still yet other aspects, when determining whether a subject is at risk of thrombosis, the subject may have previously experienced at least one thrombotic event and is suffering from SLE. In yet still other aspects, when determining whether a subject is at risk of thrombosis, the subject may have previously experienced at least one thrombotic event and is suffering from antiphospholipid syndrome. In yet still other aspects, when determining whether a subject is at risk of thrombosis, the subject may have previously experienced at least one thrombotic event and is suffering from antiphospholipid syndrome and SLE. In still other aspects, when determining whether a subject is at risk of malignancy or cancer, the subject may never have had a malignancy or cancer. In still yet other embodiments, when determining whether a subject is at risk of malignancy, the subject may have previously had at least one malignancy or cancer. In still other aspects, when determining whether a subject is at risk of malignancy or cancer, the subject may previously never had a malignancy or cancer and is suffering from SLE. In still yet other embodiments, when determining whether a subject is at risk of malignancy or cancer, the subject may have previously had at least one malignancy or cancer and is suffering from SLE. In still other aspects, when determining whether a subject is at risk of malignancy or cancer, the subject may never have had a malignancy or cancer and the subject is suffering from antiphospholipid syndrome. In still yet other embodiments, when determining whether a subject is at risk of malignancy or cancer, the subject may have previously had at least one malignancy or cancer and is suffering from antiphospholipid syndrome. In still other aspects, when determining whether a subject is at risk of malignancy or cancer, the subject may never have had previously have a malignancy or cancer and is suffering from SLE and antiphospholipid syndrome. In still yet other embodiments, when determining whether a subject is at risk of malignancy or cancer, the subject may previously have had at least one malignancy or cancer and is suffering from SLE and antiphospholipid syndrome. In still yet other aspects, the methods or assays described herein can be used as an aid in the treatment of a SLE subject determined to be at risk of thrombosis, malignancy or cancer, death, or any combination thereof. For example, the methods or assays described herein can be used in conjunction with clinical presentation and other laboratory tests to aid in determining whether a subject is at risk of thrombosis, malignancy or cancer, death, or any combination thereof. In some aspects, the subject is suffering from SLE. In some aspects, the subject is suffering from antiphospholipid syndrome. In other aspects, the subject is not suffering from SLE and/or antiphospholipid syndrome. In still other aspects, the subject has previously never experienced a thrombotic event and thus is at risk of experiencing a “new” thrombotic event. In yet still other aspects, the subject has previously experienced at least one thrombotic event and thus is at risk of experiencing a “recurrent” thrombotic event. In yet still other aspects, the subject has previously never experienced a thrombotic event but is suffering from SLE. In yet still other aspects, the subject has previously never experienced at least one thrombotic event and is suffering from SLE and antiphospholipid syndrome. In still yet other aspects, the subject has previously experienced at least one thrombotic event and is suffering from SLE. In still yet other aspects, the subject has previously experienced at least one thrombotic event and is suffering from antiphospholipid syndrome. In yet still other aspects, the subject has previously experienced at least one thrombotic event and is suffering from SLE and antiphospholipid syndrome. In still other aspects, the subject has previously never experienced a malignancy or cancer. In still other aspects, the subject has previously experienced at least one malignancy or cancer. In still other aspects, the subject has previously never experienced a malignancy or cancer and is suffering from SLE. In yet still other aspects, the subject has previously never experienced a malignancy or cancer and is suffering from antiphospholipid syndrome. In still yet further aspects, the subject has previously never experienced a malignancy or cancer and is suffering from antiphospholipid syndrome and SLE. In still other aspects, the subject has previously experienced at least one malignancy or cancer and is suffering from SLE. In yet still other aspects, the subject has previously experienced a malignancy or cancer and is suffering from antiphospholipid syndrome. In still yet further aspects, the subject has previously experienced a malignancy or cancer and is suffering from antiphospholipid syndrome and SLE.

In some aspects, the methods or assays described herein can be used in conjunction with clinical presentation and other laboratory tests to aid in the treatment of a subject who is at risk of thrombosis, malignancy or cancer, death or any combination thereof. In some aspects, the subject that is treated is suffering from SLE. In other aspects, the subject that is treated is suffering from antiphospholipid syndrome. In yet other aspects, the subject that is treated is suffering from SLE and antiphospholipid syndrome. In still yet other aspects, the subject that is treated is not suffering from SLE and/or antiphospholipid syndrome. In some aspects the subject that is treated has never experienced a previous thrombotic event (i.e., is at risk for experiencing a new thrombotic event). In other aspects, the subject that is treated has experienced at least one thrombotic event (i.e., is at risk for experiencing one or more recurrent thrombotic events). In still yet other aspects, the subject that is treated has never had a malignancy or cancer. In still yet further aspects, that subject that is treated has had at least one malignancy or cancer. In some aspects, the subject that is treated has never experienced a previous thrombotic event and is suffering from SLE. In still other aspects, the subject that is treated has never experienced a previous thrombotic event and is suffering from antiphospholipid syndrome. In still yet other aspects, the subject that is treated has never experienced a previous thrombotic event and is suffering from SLE and antiphospholipid syndrome. In other aspects, the subject that is treated has experienced at least one thrombotic event and is suffering from SLE. In other aspects, the subject that is treated has experienced at least one thrombotic event and is suffering from antiphospholipid syndrome. In still yet other aspects, the subject that is treated has experienced at least one thrombotic event and is suffering from SLE and antiphospholipid syndrome. In still yet other aspects, the subject that is treated has never had a malignancy or cancer and is suffering from SLE. In still yet other aspects, the subject that is treated has never had a malignancy or cancer and is suffering from antiphospholipid syndrome. In still yet other aspects, the subject that is treated has never had a malignancy or cancer and is suffering from SLE and antiphospholipid syndrome. In still yet further aspects, the subject that is treated has had at least one malignancy or cancer and is suffering from SLE. In still yet further aspects, the subject that is treated has had at least one malignancy or cancer and is suffering from antiphospholipid syndrome. In still yet further aspects, the subject that is treated has at least one malignancy or cancer and is suffering from SLE and antiphospholipid syndrome.

Any method or assay for detecting the presence of at least one anti-TFAM antibody (such as an anti-human TFAM antibody, such as an IgG, IgA, IgD, IgE, and/or IgM antibody) or measuring or determining the concentration (e.g., level or amount) of at least one anti-TFAM antibody (such as an anti-human TFAM antibody) can be used in the methods described herein. In some embodiments, the amount or concentration of at least one anti-TFAM antibody (such as at least one anti-human TFAM antibody) can be obtained using any assay known in the art, such as, an immunoassay, a clinical chemistry assay, a single molecule detection assay, a lateral flow assay, cytometry, mass spectroscopy, or any combinations thereof.

In some embodiments, detecting the presence of or measuring the quantity, amount, level or concentration of at least anti-TFAM antibody (e.g., such as an anti-human TFAM antibody such as an autoantibody) in at least one biological sample obtained from a subject is obtained by performing an immunoassay. For example, a biological sample suspected of containing at least one anti-TFAM antibody (such as an anti-human TFAM antibody), is brought into contact with at least one first specific binding partner (e.g., capture reagent) which comprises a polypeptide comprising or consisting of amino acids 43 to 246 of mature human TFAM (also referred to herein as at least one first capture polypeptide) to form a first mixture. In some aspects, the mixture is allowed to incubate at a temperature of from about 2° C. to about 45° C. for a period from at least about one (1) minute to about eighteen (18) hours to allow the formation of at least one first specific binding partner-anti-TFAM antibody complex. At least one type of second specific binding partner comprising at least one detectable label (e.g., a detection reagent), can be added to the first mixture at the same time as the at least one first specific binding partner, or after the formation of the at least one specific binding partner-anti-TFAM antibody complex to form a second mixture to produce one or more first complexes comprising the at least one first specific binding partner-anti-TFAM antibody-at least one second specific binding partner. The signal from the detectable label from the first complexes can then be assessed using routine techniques known in the art to determine the presence or the amount or concentration of at least one anti-TFAM antibody (e.g., such as at least one anti-human TFAM antibody) in the sample.

In some aspects, when the amount or concentration of at least one anti-TFAM antibody (e.g. at least one anti-human TFAM antibody) is determined in the biological sample, the subject can be determined or identified to be at risk of thrombosis and/or for malignancy and/or death if the amount of first complexes comprising the at least one first specific binding partner-anti-TFAM antibody-at least one second specific binding partner in the biological sample is higher than a reference level. In other aspects, when the amount or concentration of at least one anti-TFAM antibody (e.g. at least one anti-human TFAM antibody) determined in the biological sample is equal to or less than the reference level, then the subject can be determined or identified not to be at risk of thrombosis and/or for malignancy and/or death.

The reference level used in the methods and assays described herein are based on the amount or concentration of first complexes comprising the at least one first specific binding partner-anti-TFAM antibody-at least one second specific binding partner determined in a control group of subjects, such as human subjects. In some aspects, the reference level is at least one standard deviation above a mean amount of first complexes detected in a control group of subjects determined using routine techniques known in the art. In other aspects, the reference level is at least one standard deviation above a mean amount of first complexes detected in a control group of subjects who do not suffer from SLE and/or antiphospholipid syndrome. In other aspects, the reference level is at least two standard deviations above a mean amount of first complexes detected in a control group of subjects determined using routine techniques known in the art. In yet other aspects, the reference level is at least two standard deviations above a mean amount of first complexes detected in a control group of subjects who do not suffer from SLE and/or phospholipid syndrome. In still yet further aspects, when the subjects are subjects who suffer from SLE, the control group of subjects are subjects who do not suffer from SLE. In other aspects, when the subjects are subjects who suffer from antiphospholipid syndrome, the control group of subjects are subjects who do not suffer antiphospholipid syndrome. In still other aspects, when the subjects who suffer from SLE and antiphospholipid syndrome, the control subjects are subjects who do not suffer from SLE and antiphospholipid syndrome. In other aspects, when the subjects are subjects who do not suffer from SLE and/or antiphospholipid, the control group of subjects are determined using routine techniques known in the art.

When an immunoassay is used, any type of immunoassay may be utilized. The immunoassay may be an enzyme-linked immunoassay (ELISA), radioimmunoassay (RIA), a competitive inhibition assay, such as forward or reverse competitive inhibition assays, a fluorescence polarization assay, or a competitive binding assay, for example. The ELISA may be a sandwich ELISA.

The use of immobilized polypeptides or fragments thereof may be incorporated into any type of assay, such as an immunoassay, as a capture reagent or capture polypeptide. The polypeptides may be immobilized onto a variety of supports, such as magnetic or chromatographic matrix particles, the surface of an assay plate (such as microtiter wells), pieces of a solid substrate material, and the like. An assay strip can be prepared by coating the polypeptide or plurality of polypeptide in an array on a solid support. This strip can then be dipped into the test biological sample and then processed quickly through washes and detection steps to generate a measurable signal, such as a colored spot.

Optionally, prior to contacting the biological sample with the at least one first capture polypeptide, the at least one first capture polypeptide can be bound to a solid support which facilitates the separation the capture polypeptide-anti-TFAM antibody-detection antibody complex from the biological sample. Any solid support known in the art can be used, including but not limited to, solid supports made out of polymeric materials in the forms of wells, tubes or beads. The mature human TFAM polypeptide (or polypeptides) can be bound to the solid support by adsorption, by covalent bonding using a chemical coupling agent or by other means known in the art, provided that such binding does not interfere with the ability of the polypeptide to bind the antibody. Moreover, if necessary, the solid support can be derivatized to allow reactivity with various functional groups on the polypeptide. Such derivatization can be performed using routine techniques known in the art.

In some aspects, the at least one type of first specific binding partner (e.g., capture reagent) or capture polypeptide comprises a mature human TFAM polypeptide. The amino acid sequence of the full-length mature human TFAM is shown in SEQ ID NO:1. In some aspects, the mature human TFAM polypeptide used in the methods described herein can be obtained using any means known in the art. For example, the mature human TFAM polypeptide used in the methods described herein can be a natural peptide isolated from human cells (e.g., such as HeLa cells), using routine techniques known in the art. In other aspects, the mature human TFAM polypeptide can be produced recombinantly using routine techniques known in the art.

In other aspects, the first specific binding partner or capture polypeptide used in the methods described is a polypeptide comprising or consisting of amino acid sequences 43 to 246 of mature human TFAM. Methods for producing a polypeptide comprising amino acid sequences 43 to 246 of mature human TFAM from the mature full-length human TFAM are well known in the art.

In some aspects, the at least one first specific binding partner or capture polypeptide comprises or consists of amino acid sequences 43 to 246 of SEQ ID NO:1. In other aspects, the at least one first specific binding partner or capture polypeptide is a fragment of amino acid sequences 43 to 246 of SEQ ID NO: 1. As used herein, the term “fragment” when used in connection with the phrase “amino acids 43 to 246 of SEQ ID NO: 1” refers to a protein or polypeptide that comprises a part that is less than the entirety of the range of amino acids 43 to 246 of SEQ ID NO:1. More specifically, fragments of amino acid sequences 43 to 246 of SEQ ID NO: 1 can have a length of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, or 203 amino acids.

In some aspects, the at least one first specific binding partner or capture polypeptide has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or at least 100% sequence identity to amino acids 43 to 246 of SEQ ID NO:1 or a fragment thereof. In still further aspects, the at least first specific binding partner or capture polypeptide has at least 80%, at least 85%, at least 90%, at least 95%, at least 99% or at least 100% sequence identity to amino acids 43 to 246 of SEQ ID NO:1 or a fragment thereof. In some aspects, the at least first specific binding partner or capture polypeptide has at least 5% sequence identity to amino acids 43 to 246 of SEQ ID NO: 1 or a fragment thereof. In some aspects, the at least first specific binding partner or capture polypeptide has at least 10% sequence identity to amino acids 43 to 246 of SEQ ID NO:1 or a fragment thereof. In some aspects, the at least first specific binding partner or capture polypeptide has at least 15% sequence identity to amino acids 43 to 246 of SEQ ID NO: 1 or a fragment thereof. In some aspects, the at least first specific binding partner or capture polypeptide has at least 20% sequence identity to amino acids 43 to 246 of SEQ ID NO:1. In some aspects, the at least first specific binding partner or capture polypeptide has at least 25% sequence identity to amino acids 43 to 246 of SEQ ID NO: 1 or a fragment thereof. In some aspects, the at least first specific binding partner or capture polypeptide has at least 30% sequence identity to amino acids 43 to 246 of SEQ ID NO: 1 or a fragment thereof. In some aspects, the at least first specific binding partner or capture polypeptide has at least 35% sequence identity to amino acids 43 to 246 of SEQ ID NO:1 or a fragment thereof. In some aspects, the at least first specific binding partner or capture polypeptide has at least 40% sequence identity to amino acids 43 to 246 of SEQ ID NO: 1 or a fragment thereof. In some aspects, the at least first specific binding partner or capture polypeptide has at least 45% sequence identity to amino acids 43 to 246 of SEQ ID NO: 1 or a fragment thereof. In some aspects, the at least first specific binding partner or capture polypeptide has at least 50% sequence identity to amino acids 43 to 246 of SEQ ID NO:1 or a fragment thereof. In some aspects, the at least first specific binding partner or capture polypeptide has at least 55% sequence identity to amino acids 43 to 246 of SEQ ID NO: 1 or a fragment thereof. In some aspects, the at least first specific binding partner or capture polypeptide has at least 60% sequence identity to amino acids 43 to 246 of SEQ ID NO: 1 or a fragment thereof. In some aspects, the at least first specific binding partner or capture polypeptide has at least 65% sequence identity to amino acids 43 to 246 of SEQ ID NO:1 or a fragment thereof. In some aspects, the at least first specific binding partner or capture polypeptide has at least 70% sequence identity to amino acids 43 to 246 of SEQ ID NO: 1 or a fragment thereof. In some aspects, the at least first specific binding partner or capture polypeptide has at least 75% sequence identity to amino acids 43 to 246 of SEQ ID NO: 1 or a fragment thereof. In some aspects, the at least first specific binding partner or capture polypeptide has at least 80% sequence identity to amino acids 43 to 246 of SEQ ID NO: 1 or a fragment thereof. In some aspects, the at least first specific binding partner or capture polypeptide has at least 85% sequence identity to amino acids 43 to 246 of SEQ ID NO: 1 or a fragment thereof. In some aspects, the at least first specific binding partner or capture polypeptide has at least 90% sequence identity to amino acids 43 to 246 of SEQ ID NO: 1 or a fragment thereof. In some aspects, the at least first specific binding partner or capture polypeptide has at least 95% sequence identity to amino acids 43 to 246 of SEQ ID NO:1 or a fragment thereof. In some aspects, the at least first specific binding partner or capture polypeptide has at least 99% sequence identity to amino acids 43 to 246 of SEQ ID NO: 1 or a fragment thereof. In some aspects, the at least first specific binding partner or capture polypeptide has at least 100% sequence identity to amino acids 43 to 246 of SEQ ID NO: 1 or a fragment thereof. In yet another aspect, the at least first specific binding partner or capture polypeptide comprises or consists of amino acids 43 to 246 of the below sequence:

MAFLRSMWGVLSALGRSGAELCTGCGSRLRSPFSFVYLPRWFSSV
LASCPKKPVSSYLRFSKEQLPIFKAQNPDAKTTELIRRIAQRWRE
LPDSKKKIYQDAYRAEWQVYKEEISRFKEQLTPSQIMSLEKEIMD
KHLKRKAMTKKKELTLLGKPKRPRSAYNVYVAERFQEAKGDSPQE
KLKTVKENWKNLSDSEKELYIQHAKEDETRYHNEMKSWEEQMIEV
GRKDLLRRTIKKQRKYGAEEC
(SEQ ID NO: 1) or a fragment thereof.

In some aspects, the at least one second specific binding partner is an anti-IgA antibody, an anti-IgD antibody, an anti-IgE antibody, an anti-IgG antibody, an anti-IgM antibody, or any combinations thereof. In some aspects, the at least one second specific binding an anti-human IgA antibody, an anti-human IgD antibody, an anti-human IgE antibody, an anti-human IgG antibody, an anti-human IgM antibody, or any combinations thereof. In some aspects, the at least one second specific binding partner further comprises at least one detectable label (detection reagent). In some embodiments, the at least one second specific binding partner is immobilized on one or more solid supports.

In some aspects, at least one anti-TFAM antibody (e.g., autoantibody) specifically binds to a first epitope on the at least first specific binding partner or capture polypeptide comprising or consisting of amino acid 43 to 246 (e.g., SEQ ID NO:1) or a fragment thereof and the second specific binding partner specifically binds to an antibody binding site or paratope (e.g., a second epitope) on the at least one anti-TFAM antibody. In some aspects, the first epitope has a length or comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, or 203 amino acids. In some aspects, the antibody binding site or paratope has a length or comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, or 225 amino acids.

In some embodiments, the method further comprises communicating the presence or amount, level or concentration of anti-TFAM antibodies in a biological sample. In some embodiments, the method comprises communicating the amount or concentration of a subject's anti-TFAM antibodies on or from at least one instrument. Suitable instruments are described herein, including point-of-care devices and non-point-of care devices that may contain a user interface that communicates by displaying the determination.

As discussed, in some embodiments, the instrument contains software to execute one or more tasks. In some embodiments, the instrument contains software to automatically determine the next appropriate step in a method as described herein. For example, the instrument may contain software that determines the amount or concentration of anti-TFAM antibodies (e.g., such as anti-human TFAM antibodies) in a biological sample obtained from a subject. The software may display this determination, such as on a graphical user interface.

In some embodiments, the instrument stores software that instructs a processor to execute a given task. In some embodiments, the software stores machine readable instructions that instruct a processor to execute a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine-readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

3. Treatment and Monitoring of Subjects Identified as Having a Certain Amount, Concentration and/or Level of at Least One Anti-TFAM Antibody

A subject identified according to the methods described above can and/or having at least one anti-TFAM antibody and/or having a certain amount, concentration and/or level of at least one anti-TFAM antibody may be treated, monitored (e.g., anti-TFAM IgG, IgA, IgD, IgE and/or IgM antibody levels monitored in the subject), treated and monitored and/or monitored and treated using routine techniques known in the art. In some aspects, the methods described herein further include treating the subject (e.g., such as a human) identified as having at least one anti-TFAM antibody and/or having a certain amount, concentration and/or level of at least one anti-TFAM antibody in one or more biological samples obtained from the subject as described in Section 2 herein. In some aspects, the subject is suffering from SLE and/or antiphospholipid syndrome.

In some aspects, when the subject is suffering from SLE, the treatment can vary depending on whether the subject is asymptomatic or experiencing mild, moderate, or severe symptoms. For instance, subjects with mild SLE may exhibit symptoms such as fatigue, joint pain, skin rashes (e.g., a butterfly rash across the cheeks), mild fever, and general malaise, or any combination thereof. Those with moderate SLE may experience increased joint inflammation, more pronounced fatigue, hair loss, or other systemic manifestations. Severe cases may present with serious complications affecting the kidneys (lupus nephritis), neurological symptoms, or hematological issues, such as anemia or thrombocytopenia.

If the subject is asymptomatic or has mild symptoms, treatment may include rest, hydration, and over-the-counter pain relievers (e.g., NSAIDs) to manage discomfort. Additionally, subjects can be advised to monitor their symptoms and maintain regular follow-ups with healthcare providers to assess any changes in their condition.

Subjects exhibiting moderate or severe symptoms of SLE may require more aggressive treatment strategies, including corticosteroids, immunosuppressants, antithrombotic agents or biologic therapies (e.g., belimumab). These subjects may also need close monitoring for potential organ involvement and adverse effects of medication, as well as supportive therapies to manage specific symptoms.

In some aspects, when the subject is identified as at risk for thrombosis or a thrombotic event, either new or recurrent, the subject can be treated with one or more pharmaceutical agents, such as, but are not limited to, low-dose aspirin, direct factor Xa inhibitors, thrombin inhibitors, PY12 inhibitors, low molecular weight inhibitors, warfarin, heparin, hydroxychloroquine, azathioprine, mycophenolate mofetil, or combinations thereof. Patients receiving these treatments should also be regularly monitored using established clinical techniques to ensure effective management of their condition. In some aspects, the subject identified as at risk for thrombosis, or a thrombotic event is suffering from SLE and/or antiphospholipid syndrome.

In other aspects, a subject may be monitored prior to or after treatment. Such monitoring involves detecting, analyzing and/or interpreting changes in the subject's anti-TFAM IgG, IgA, IgD. IgE and/or IgM antibody levels over the course of time. For example, depending on a subject's TFAM IgM antibody level, a subject may be monitored prior to receiving any treatment to gauge whether the subject is at risk of thrombosis or malignancy. During the course of the monitoring, if the subject's TEAM IgM antibody levels increase, treatment can be commenced. Likewise, during treatment, a subject's TFAM IgM and IgG levels can be monitored. In some aspects, the subject is suffering from SLE and/or antiphospholipid syndrome.

### 4. Samples and Controls

As used herein, “sample”, “test sample”, “biological sample” refer to fluid sample containing or suspected of containing an anti-TFAM antibody, such as an anti-TFAM (IgG, IgA, IgD. IgE and/or IgM) antibody. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing at least one anti-TFAM antibody may be assayed directly. In a particular example, the source of an anti-TFAM antibody is a mammalian (e.g., human) bodily substance (e.g., bodily fluid, blood such as whole blood (including, for example, capillary blood, venous blood, etc.), anal swab specimens, serum, plasma, urine, saliva, sweat, sputum, semen, mucus, nasal mucus, lacrimal fluid, lymph fluid, amniotic fluid, interstitial fluid, lower respiratory specimens such as, but not limited to, sputum, endotracheal aspirate or bronchoalveolar lavage, cerebrospinal fluid, feces, tissue, organ, one or more dried blood spots, or the like). Tissues may include, but are not limited to oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

In some cases, the sample may undergo pre-analytical processing or pre-treatment. Pre-analytical processing may offer additional functionality such as nonspecific protein removal and/or effective yet cheaply implementable mixing functionality. General methods of pre-analytical processing may include the use of electrokinetic trapping, AC electrokinetics, surface acoustic waves, isotachophoresis, dielectrophoresis, electrophoresis, or other pre-concentration techniques known in the art.

### 5. Kits

Provided herein is a kit, which may be used to determine the presence or the amount or concentration of at least one anti-TFAM antibody (e.g., at least one anti-human antibody) in a biological sample obtained from a subject. The kit can further comprise at least one capture reagent and at least one detection reagent. In yet further embodiments, kit can comprise instructions for assaying the test sample for at least one biomarker by immunoassay, e.g., chemiluminescent microparticle immunoassay, a clinical chemistry assay, a lateral flow assay, a mass spectroscopy assay, or any other assay known in the art. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. Alternatively or additionally, the kit can comprise a calibrator or control for the at least one biomarker and/or at least one container (e.g., tube, microtiter plates or strips, which can be already coated with the relevant biomarker for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

In some embodiments, the kit is useful for assaying a biological sample for at least one anti-TFAM antibody (e.g., such as at least one anti-human TFAM antibody). The kit comprises at least one component for assaying the test sample for one or more anti-TFAM antibodies (e.g. instructions for assaying the biological sample. For example, the kit can comprise instructions for assaying the biological sample for one or more anti-TFAM antibodies by any type of assay, such as an immunoassay, e.g., chemiluminescent microparticle immunoassay. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like.

The at least one component may include at least one composition comprising one or more isolated mature human TFAM polypeptides or fragments thereof that specifically bind to anti-TFAM antibodies, such as anti-human TFAM antibodies.

Alternatively or additionally, the kit can comprise a calibrator or control, e.g., purified, and optionally lyophilized, anti-TFAM antibodies (such as anti-TFAM human antibodies) and/or at least one container (e.g., tube, microtiter plates or strips, which can be already coated with an a mature human TFAM polypeptide comprising or consisting of amino acids 43 to 246 of SEQ ID NO: 1) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying one or more anti-TFAM antibodies. The reference standards may be employed to establish standard curves for interpolation and/or extrapolation of anti-TFAM antibody concentrations.

Any antibodies, which are provided in the kit, specific for anti-TFAM antibodies, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., anti-TFAM antibodies) or reagents for detecting the analyte (e.g., anti-TFAM antibodies). The polypeptides, antibodies, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pre-treatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

The present disclosure has multiple aspects, illustrated by the following non-limited examples.

### EXAMPLES

### Methods

Sex as a biological variable. Male and female subjects were included, although females predominate (9:1 female:male ratio) due to the disease demographics.

Study design and patients. Exploratory sample of 9 healthy controls and 22 SLE patients, and a sample of 98 health controls (median age [IQR] 37 (30, 45 years; female sex 71/98, 72%) and 158 SLE patients from the “Study of biological Pathways, Disease Activity and Response markers in patients with Systemic Lupus Erythematosus” (SPARE) cohort were used (Zollars et al., 2015; Zollars et al., 2016). Briefly, adult patients (age 18 to 75 years-old) who met the definition of SLE per the revised American College of Rheumatology classification criteria were enrolled (Hochberg, 1997). Patients were treated according to standard clinical practice. Disease activity was assessed using the Safety of Estrogens in Lupus Erythematosus: National Assessment (SELENA) version of the Systemic Lupus Erythematosus Disease Activity Index (SLEDAI) (Petri et al., 2005) and physician global assessment (PGA) (Petri et al., 1992). C3, C4, anti-dsDNA (Crithidia), complete blood cell count, and urinalysis were performed at every visit. Study participants also underwent whole blood gene expression analysis using the Affymetrix Gene Chip HT HG-U133+ (Zollars et al., 2015; Zollars et al., 2016), as well as quantification of circulating IFN-I, IFN-II and IFN-III activity levels (Gomez-Banuelos et al., 2024). Antibodies to TFAM were also measured in 26 patients with rheumatoid arthritis (RA), 40 with dermatomyositis (DM) and 50 with PAPS.

TFAM cloning and protein expression. cDNA encoding human mature TFAM (amino acids 43-246) was synthesized using RNA from human PBMCs and cloned into pET-28a(+). Recombinant N-terminal his-tagged TFAM was expressed in E. coli BL21 (DE3) and purified by Ni-NTA affinity chromatography. TFAM structure was predicted using AlphaFold Protein Structure Database.

Detection of antibodies to TFAM. Anti-TFAM antibodies were measured in serum/plasma using an in-house developed ELISA. Briefly, Nunc Maxisorp plates were coated with 200 ng/well of recombinant TFAM. The plates were blocked for one hour with phosphate buffered saline plus 0.1% Tween-20 (PBST) with 3% non-fat milk. Serum/plasma was diluted 1:1000 in PBST plus 1% non-fat milk and assayed in duplicate using antigen-conjugated plates and plates without antigen for background subtraction. For competition assays, diluted SLE serum/plasma was pre-incubated with 10 μg/mL of human recombinant human recombinant high-mobility group box 1 (HMGB1) (Sino Biological) or B2-glycoprotein I (β2GPI) purified from human plasma (Arotec Diagnostics) for 1 hour at room temperature. Horseradish peroxidase (HRP)-conjugated goat anti-human IgG was used as a secondary antibody (Diluted at 1:10,000 in PBST 1% nonfat milk). TFAM antibody arbitrary units (AU) were calculated using a standard curve made using serial dilutions of serum from a high-titre SLE patient. Anti-TFAM positivity cutoff was set as the mean+2SD of healthy control anti-TFAM antibodies (in AU) from a healthy control.

Indirect immunofluorescence. Peripheral blood human neutrophils were purified as previously described (Darrah et al., 2012), attached to microscope slides using HistoGrip™ (cat. 008050 ThermoFisher Scientific), fixed with 4% paraformaldehyde and permeabilized with acetone. After blocking with 2% bovine serum albumin (BSA) and incubation with SLE serum diluted at 1:100 in PBS or commercial anti-TFAM antibody (clone C9, cat. sc-376672, Santa Cruz Biotechnologies) at 1:10, human IgG and commercial anti-TFAM antibodies were visualized using Alexa Fluor 488-conjugated goat anti-human IgG or Alexa Fluor 594-conjugated rabbit anti-mouse antibodies, respectively, DNA was detected using 4′,6-diamidino-2-phenyl-indole (DAPI). In addition, TFAM detection using the commercial anti-TFAM antibody (clone C9) and anti-TFAM positive SLE sera was performed using commercially available Hep-2 cell slides (NOVA Lie Hep-2 ANA Kit with DAPI, cat. 708102; Werfern) according to manufacturer's instructions. For Hep-2 testing, serum was diluted at 1:80 and the commercial antibody at 1:10. Human IgG and commercial anti-TFAM antibodies were visualized using Alexa Fluor 488-conjugated goat anti-human IgG or Alexa Fluor 594-conjugated rabbit anti-mouse antibodies, respectively. For competition assays, diluted SLE serum was pre-incubated with 40 μg/mL of mature human recombinant TFAM for 1 hour at room temperature.

Gene expression analyses. Gene expression analysis from the SPARE cohort was previously described (Zollars et al., 2016). CEL files were subjected to robust multi-array average (RMA) background correction, and quantile normalization, using the oligo R package (Banchereau et al., 2016). To select only expressed genes in whole blood, transcripts that had a raw signal <100 in less than 10% of samples were filtered out with the genefilter R package. All calculations and analyses were performed using R (ver 4.0.2) and Bioconductor (ver 3.13) (Huber et al., 2015).

Feature selection using the fisher score algorithm and PCA analysis. To select the most relevant transcripts to distinguish patients positive or negative for anti-TFAM antibodies, thrombotic events, or anti-dsDNA antibodies, an algorithm known as the Fisher score was employed (Li et al., 2019). Briefly, a Fisher score was calculated for each individual transcript by considering the number of samples, mean and standard deviation on each category (e.g., anti-TFAM positive or anti-TFAM negative) and each transcript was then ranked according to its score. For the principal component analysis (PCA), the top 1000 genes were selected to identify biologically relevant pathways using the Fisher score algorithm (Li et al., 2019). Fisher scores were calculated using the Rdimtools R package (You et al., 2022). PCA was performed using base R functions.

Gene set enrichment analyses. To perform gene set enrichment analyses, the transcripts with the highest loading on the anti-TFAM antibodies, thrombosis and anti-dsDNA antibodies PC1 were determined by calculating the Pearson's r correlation coefficient. P values were adjusted with the Benjamini-Hochberg method. Transcripts with an adjusted p value ≤0.01 were considered as significantly correlated. In order to compare the anti-TFAM, thrombosis and anti-dsDNA related transcriptomes, gene set enrichment analysis was performed using the metanalysis function of Metascape (Zhou et al., 2019).

Statistical analyses. Continous variables were compared using Student's T test and ANOVA test with Tukey's post hoc test as indicated. The Mann-Whitney's U test and Kruskal-Wallis's tests were used for group-wise comparisons of non-normally distributed variables Fisher's exact test and χ² tests were used for univariate analysis on SPARE cohort variables, as appropriate. The exact2×2 package in R version 3.5.1 was used for binary variables to obtain P value, odds ratio (OR), and 95% CI. Correlations between variables were calculated using Pearson's r. Comparisons of paired samples were performed using Wilcoxon signed-rank test. Multivariate analyses were carried out using multivariate logistic regression. Statistical significance was set at p<0.05. The statistical analyses were carried out with the R software version 4.3.3.

### Results

### TFAM is an Autoantigen in SLE.

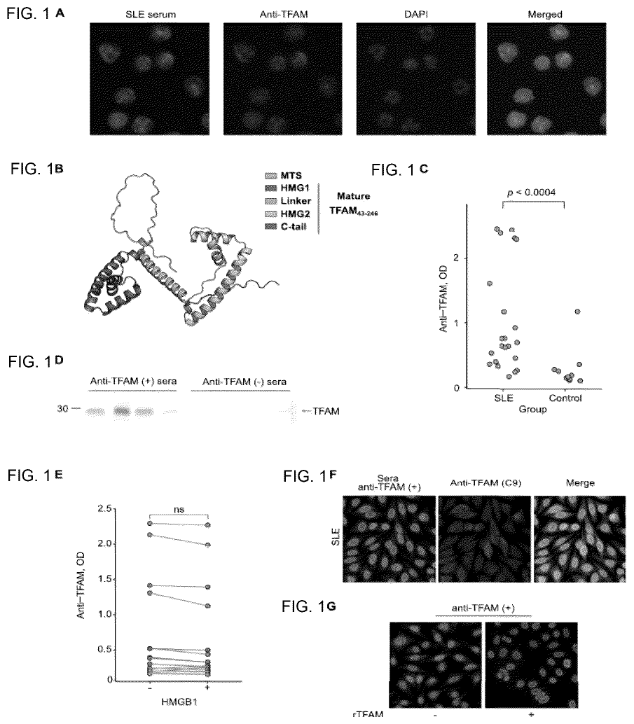

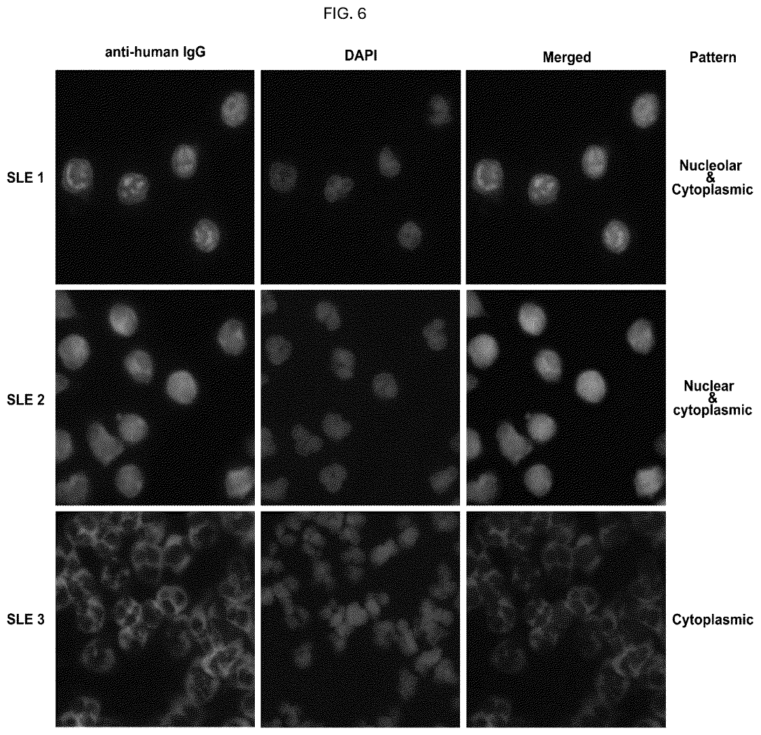

Autoantibodies in SLE bind to different antigens in human neutrophils, generating distinct cellular patterns when analyzed by indirect immunofluorescence, including nucleolar, nuclear and cytoplasmic patterns (FIG. 6). Coincidentally, while working on parallel studies with neutrophils and TFAM, it was noticed that commercial antibodies to TFAM generate a cellular pattern similar to SLE sera targeting cytoplasmic antigens (FIG. 1A), leading to the hypothesis that TFAM is an autoantigen in SLE. TFAM is a nuclear-encoded protein of 204 amino acids (24 kDa). It is translated as a preprotein containing a mitochondrial targeting sequence (MTS; amino acids 1-42) that is cleaved upon mitochondrial import (FIG. 1B). Mature TFAM (amino acids 43-246) consist of 2 HMG boxes separated by a linker and followed by a short tail at the C-terminus (FIG. 1B).

To address whether TFAM is a target of autoantibodies in SLE, an ELISA assay was developed using mature human recombinant TFAM as a substrate and screened sera from an exploratory cohort sample of patients with SLE (n=22) and healthy controls (n=9). Patients with SLE had significantly elevated serum levels of anti-TFAM antibodies compared with healthy controls (P<0.0004) (FIG. 1C). Notably, SLE sera associated with a cytoplasmic pattern in neutrophils (either alone or concomitant with a nuclear pattern) (FIG. 1A and FIG. 6) were those with the highest levels of anti-TFAM antibodies detected by ELISA. It was further confirmed that the presence of antibodies to TFAM in SLE by immunoblotting (FIG. 1D), and that these antibodies were not cross-reactive with HMGB1 (FIG. 1E), a HMG protein analogous to TFAM that is also an autoantigen in SLE (Uesugi et al., 1998). In Hep-2 cells, anti-TFAM-positive SLE serum colocalised with TFAM staining, which showed a cytoplasmic reticular pattern consistent with the AC-21 antinuclear antibody pattern (FIG. 1F). However, unlike in neutrophils (FIG. 1A), TFAM also showed some nuclear localisation in Hep-2 cells, which might be explained by the fixation/permeabilisation method or the expression of a TFAM variant lacking the MTS. The antibody pattern that colocalised with TFAM was completely blocked by preincubating SLE serum with mature recombinant TFAM (FIG. 1G).

Antibodies to TFAM are Associated with APS and Thrombotic Events in SLE.

To define clinical features and transcriptional pathways associated with anti-TFAM antibodies in SLE, samples from the SPARE cohort for which extensive clinical and serologic variables were available were studied, as well as whole blood gene expression data (Gomez-Banuelos et al., 2023; Zollars et al., 2016). Demographic, clinical and laboratory features of the SLE cohort are summarized in Table 1.

TABLE 1
Clinical, laboratory and disease activity associations
at time of visit according to anti-TFAM positivity.

| | Anti-TFAM | | |
|---|---|---|---|
| | Negative | Positive | p |
| | n = 108 (100%) | n = 47 (100%) | value |
| Age, yrs | 48.6(15.2) | 55.5(14.9) | 0.01 |
| Disease duration, yrs | 18.2(11.1) | 24.4(12.1) | 0.003 |
| Female sex | 104(95) | 48(94) | 0.701 |
| Black | 41(87.2) | 57(91.9) | 0.417 |
| Weight, kg | 168.3(41.266) | 179.8(51.057) | 0.18 |
| SBP, mmHg | 124.7(16.986) | 126.7(23.766) | 0.60 |
| DBP, mmHg | 72.8(10.754) | 73.1(11.796) | 0.90 |
| SLEDAI | 2(2.583) | 3(3.196) | 0.04 |
| Neurologic, n (%) | | | |
| Seizure | 0(0) | 0(0) | 1 |
| Psychosis | 0(0) | 0(0) | 1 |
| Organic Brain Syndrome | 1(0.9) | 0(0) | 1 |
| Visual disturbance | 0(0) | 0(0) | 1 |
| Cranial nerve disorder | 0(0) | 0(0) | 1 |
| Cerebral vascular | 0(0) | 0(0) | 1 |
| accident | | | |
| Lupus Headache | 0(0) | 0(0) | 1 |
| Vasculitis, n (%) | 3(2.8) | 2(4.3) | 0.639 |
| Renal, n (%) | | | |
| Urinary Casts | 0(0) | 0(0) | 1 |
| Hematuria | 2(1.9) | 1(2.1) | 1 |
| Proteinuria | 2(1.9) | 3(6.4) | 0.164 |
| Pyuria | 2(1.9) | 0(0) | 1 |
| Musculoskeletal, n (%) | | | |
| Arthritis | 6(5.6) | 3(6.4) | 1 |
| Myositis | 0(0) | 0(0) | 1 |
| Immunological, n (%) | | | |
| Low Complement | 11(10.2) | 9(19.1) | 0.19 |
| Anti-DNA | 20(18.5) | 17(36.2) | 0.024 |
| Skin, n (%) | | | |
| Rash | 7(6.5) | 5(10.6) | 0.513 |
| Alopecia | 20(18.5) | 17(36.2) | 0.024 |
| Mucosal Ulcers | 5(4.6) | 0(0) | 0.323 |
| Serositis, n (%) | | | |
| Pleurisy | 2(1.9) | 1(2.1) | 1 |
| Pericarditis | 0(0) | 0(0) | 1 |
| Hematological, n (%) | | | |
| Thrombocytopenia | 1(0.9) | 1(2.1) | 0.516 |
| Leukopenia | 1(0.9) | 0(0) | 1 |
| Constitutional, n (%) | | | |
| Fever | 0(0) | 0(0) | 1 |
| Russell's Viper Venom | 37(7.683) | 43.7(22.756) | 0.06 |
| Test, seconds | | | |
| Hemoglobin, g/dL | 12.5(1.299) | 12.2(1.501) | 0.23 |
| White blood | 6(2.182) | 5.7(2.588) | 0.52 |
| cells, K/mm³ | | | |
| Platelets, K/mm³ | 254.9(78.941) | 259.6(86.662) | 0.75 |
| Erythrocyte | 24.7(20.453) | 42.6(30.833) | <0.001 |
| sedimentation | | | |
| rate, mm/hr | | | |
| hsCRP, mg/L | 3.9(5.703) | 6(10.838) | 0.51 |
| C3, mg/dL | 125(36.602) | 127(43.63) | 0.78 |
| C4, mg/dL | 24.2(10.043) | 23.8(11.565) | 0.81 |
| Anti-DNA, AU | 21.1(76.323) | 75.3(180.143) | 0.05 |
| Anticardiolipin | | | |
| antibodies | | | |
| IgG, GPL units | 7.3(7.692) | 10.4(15.17) | 0.19 |
| IgM. MPL units | 8.6(8.046) | 9.4(5.128) | 0.44 |
| IgA, APL units | 4.1(2.679) | 5.7(5.525) | 0.07 |

Continuous variables are summarized as mean (SD). Associations between categorical variables were determined by chi-square or Fisher's exact tests as appropriate. Comparisons between continuous variables were done using Student's T test. SBP, systolic blood pressure. DBP, diastolic blood pressure. SLEDAI, Systemic Lupus Erythematosus disease activity index. GPL, IgG Phospholipid Units. MPL, IgM Phospholipid Units. APL, IgA Phospholipid Unit.

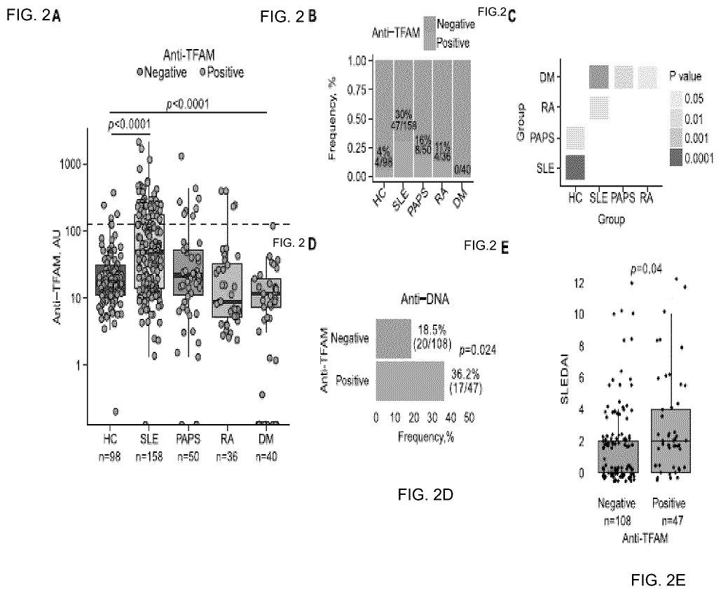

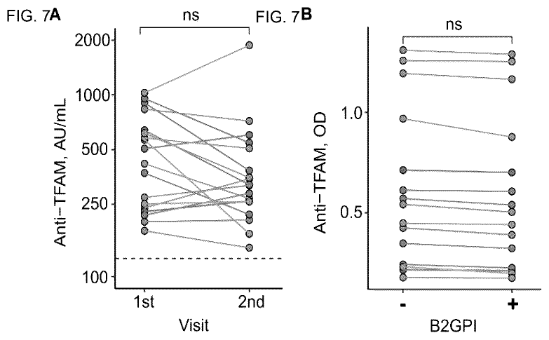

The presence of antibodies to TFAM were also measured in serum from patients with PAPS, RA, and DM. Among these different disease groups, only patients with SLE had significantly higher levels of anti-TFAM antibodies than did healthy controls [mean (SD), 126.1 (236.4) vs. 32.2 (50.5), p<0.0001] (FIG. 2A). After determining a cut-off level of two standard deviations above the mean anti-TFAM antibody level in healthy sera, 30.3% (48/158) of SLE patients were positive for antibodies to TFAM compared to 4% (4/98) in healthy controls (P<0.0001) (FIG. 2B and FIG. 2C). In addition to SLE, PAPS was linked with an increased frequency of anti-TFAM antibodies, 16% (8/50) vs. healthy controls (P<0.05). Anti-TFAM antibodies were not significantly increased in patients with RA or DM compared to healthy controls (FIG. 2A-FIG. 2C). In SLE, anti-TFAM antibodies were stable over time. Although anti-TFAM antibodies varied between visits (intraclass correlation 0.622, P=0.002), none of the samples were negative for the autoantibody, and the mean anti-TFAM levels were not statistically different between visits (mean±standard deviation (SD), 500±282 AU/mL vs 432±392 AU/mL, P-0.555 (FIG. 7A).

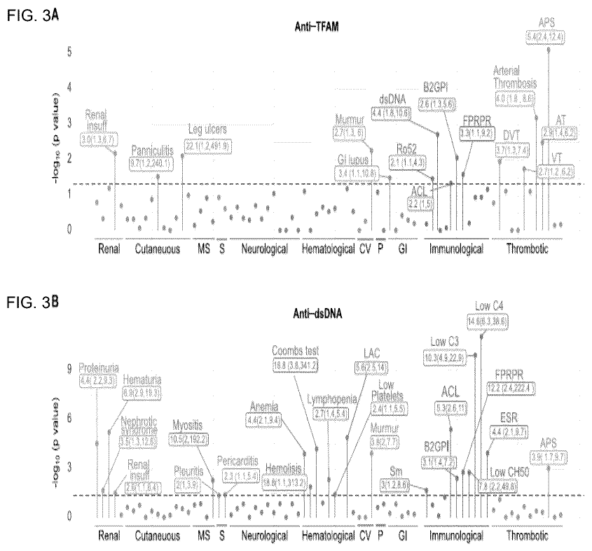

Since mtDNA has been implicated in the production of anti-dsDNA antibodies in SLE (Reimer et al., 1984), and TFAM forms a complex with mtDNA (Fisher et al., 1985; Parisi et al., 1991; Kukat et al., 2013), it was anticipated that antibodies to TFAM would be mechanistically linked to anti-dsDNA antibodies and disease activity in SLE. Surprisingly, however, only 36.2% (17/47) of anti-TFAM positive patients were also positive to anti-dsDNA antibodies (FIG. 2D), and there was a mild correlation between SLEDAI score (FIG. 2E, and Table 1). Instead, anti-TFAM antibodies were significantly associated with renal insufficiency (but not active nephritis) and features of APS (FIG. 3A, and Tables 1 and 2), in contrast to anti-dsDNA antibodies, which showed prominent association with features linked to IC deposition, such as nephritis and low complement (FIG. 3B).

TABLE 2
Clinical and laboratory associations present during the clinical
course of SLE according to anti-TFAM antibody positivity.

| | Anti-TFAM | | |
|---|---|---|---|
| | Negative | Positive | p. |
| Clinical | n = 108 (100%) | n = 47 (100%) | value |
| Deceased | 1(0.9) | 5(10.4) | 0.011 |
| Constitutional | | | |
| Fever | 35(32.1) | 20(41.7) | 0.278 |
| Lymphadenopathy | 42(38.5) | 23(47.9) | 0.295 |
| Kidney | | | |
| Proteinuria | 47(43.1) | 27(56.2) | 0.165 |
| Nephrotic syndrome | 15(13.8) | 9(18.8) | 0.473 |
| Hematuria | 29(26.6) | 20(41.7) | 0.065 |
| Renal insufficiency | 18(16.5) | 18(37.5) | 0.007 |
| Renal failure | 3(2.8) | 4(8.3) | 0.202 |
| Cutaneous | | | |
| Photosensitivity | 59(54.1) | 29(60.4) | 0.49 |
| Malar rash | 60(55) | 30(62.5) | 0.484 |
| Mouth ulcers | 63(57.8) | 27(56.2) | 0.863 |
| SCLE | 5(4.6) | 4(8.3) | 0.457 |
| Discoid | 19(17.4) | 14(29.2) | 0.136 |
| Panniculitis | 1(0.9) | 4(8.3) | 0.031 |
| Alopecia | 70(64.2) | 32(66.7) | 0.857 |
| Raynaud | 62(56.9) | 28(58.3) | 1 |
| Vasculitis | 13(11.9) | 8(16.7) | 0.45 |
| Leg ulcers | 0(0) | 4(8.3) | 0.008 |
| Livedo | 34(31.2) | 22(45.8) | 0.103 |
| Musculoskeletal | | | |
| Arthralgia | 101(92.7) | 46(95.8) | 0.725 |
| Arthritis | 84(77.1) | 41(85.4) | 0.286 |
| Joint erosions | 0(0) | 2(5.3) | 0.124 |
| Myositis | 10(9.2) | 6(12.5) | 0.571 |
| Serositis | | | |
| Pleuritis | 55(50.5) | 31(64.6) | 0.119 |
| Pericarditis | 26(24.1) | 16(33.3) | 0.245 |
| Neurological | | | |
| Seizure | 7(6.4) | 1(2.1) | 0.436 |
| Psychosis | 1(0.9) | 2(4.2) | 0.222 |
| Organic brain syndrome | 5(4.6) | 4(8.3) | 0.457 |
| Meningitis | 1(0.9) | 1(2.1) | 0.519 |
| Stroke | 3(2.8) | 4(8.3) | 0.202 |
| Depression | 46(42.2) | 17(35.4) | 0.482 |
| Headache | 8(7.3) | 7(14.6) | 0.236 |
| Mononeuritis multiplex | 0(0) | 2(4.2) | 0.092 |
| Cognitive impairment | 10(9.2) | 4(8.3) | 1 |
| Optic neuritis | 0(0) | 0(0) | 1 |
| Cranial neuropathy | 4(3.7) | 1(2.1) | 1 |
| Peripheral neuropathy | 7(6.4) | 1(2.1) | 0.436 |
| Transverse myelitis | 3(2.8) | 1(2.1) | 1 |
| Hematological | | | |
| Anemia | 76(69.7) | 40(83.3) | 0.08 |
| Hemolytic anemia | 7(6.4) | 3(6.2) | 1 |
| Coombs test | 15(13.8) | 10(20.8) | 0.343 |
| Leukopenia | 44(40.4) | 25(52.1) | 0.222 |
| Lymphopenia | 44(40.4) | 24(50) | 0.296 |
| Thrombocytopenia | 26(23.9) | 16(33.3) | 0.243 |
| Lupus Anticoagulant | 32(29.4) | 22(45.8) | 0.068 |
| Cardiac | | | |
| Myocarditis | 2(1.8) | 1(2.1) | 1 |
| Libman-Sacks | 3(2.8) | 0(0) | 0.553 |
| Murmur | 51(46.8) | 34(70.8) | 0.006 |
| Pulmonary | | | |
| Lung fibrosis | 12(11) | 11(22.9) | 0.084 |
| Pulmonary hypertension | 15(13.8) | 7(14.6) | 1 |
| Gastrointestinal | | | |
| GI lupus | 6(5.5) | 8(16.7) | 0.033 |
| Hepatomegaly | 4(3.7) | 1(2.1) | 1 |
| Abnormal liver function | 47(43.1) | 25(52.1) | 0.385 |
| tests | | | |
| Splenomegaly | 1(0.9) | 1(2.1) | 0.519 |
| Pancreatitis | 3(2.8) | 2(4.2) | 0.642 |
| Sjogren syndrome | 32(29.4) | 12(25) | 0.7 |
| Immunological | | | |
| Anti-DNA | 58(53.2) | 40(83.3) | 0.002 |
| Anti-Sm | 23(21.1) | 8(16.7) | 0.664 |
| Anti-Ro52 | 37(33.9) | 25(52.1) | 0.035 |
| Anti-La | 17(15.6) | 7(14.6) | 1 |
| Anti-RNP | 28(25.7) | 13(27.1) | 0.846 |
| Anticardiolipin | 66(60.6) | 37(77.1) | 0.047 |
| Anti-B2GPI | 27(25) | 22(46.8) | 0.009 |
| FPRPR | 8(7.3) | 10(20.8) | 0.026 |
| Low CH50 | 15(13.8) | 8(16.7) | 0.631 |
| Low C3 | 55(50.5) | 31(64.6) | 0.119 |
| Low C4 | 45(41.3) | 27(56.2) | 0.117 |
| Elevated ESR | 77(70.6) | 41(85.4) | 0.07 |
| Thrombotic | | | |
| Transient ischemic attack | 2(1.9) | 3(6.2) | 0.17 |
| Deep vein thrombosis | 13(11.9) | 14(29.2) | 0.012 |
| Cerebrovascular accident | 11(10.1) | 10(20.8) | 0.079 |
| Myocardial infarction | 2(1.8) | 1(2.1) | 1 |
| Digital necrosis | 4(3.7) | 2(4.2) | 1 |
| Venous Thrombosis | 17(15.6) | 16(33.3) | 0.018 |
| Arterial Thrombosis | 17(15.7) | 20(42.5) | <0.001 |
| Any thrombotic event | 29(26.9) | 25(53.1) | 0.003 |
| Antiphospholipid Syndrome | 20(18.5) | 26(55.3) | <0.001 |
| Preeclampsia | 4(3.7) | 4(8.5) | 0.246 |
| Miscarriages | 22 | 13 | 0.399 |

All variables are summarized as n (%) unless otherwise indicated. Associations between variables were done using chi-square or Fisher's exact test. SCLE, Subacute cutaneous lupus erythematosus. Anti-B2GPI, Anti-β2 Glycoprotein I. FPRPR, false positive rapid plasma regain test. ESR, erythrocyte sedimentation rate.

Notably, SLE patients positive for anti-TFAM antibodies were more likely to have a history of leg ulcers, cardiac murmur, any thrombotic event, and be classified with APS [OR (95% CI), 22.1 (1.2, 491.9), 2.7 (1.3, 6.0), 2.9 (1.4, 6.2), and 5.4 (2.5, 11.9), respectively] (FIG. 3A, and Table 2). Anti-TFAM antibodies were also associated with increased frequency of antibodies to β2GPI, cardiolipin and dsDNA [OR (95% CI), 2.6 (1.3, 5.6), 2.2 (1.0, 5.0), and 4.4 (1.8, 10.6), respectively]. Given the strong association between APS and anti-TFAM antibodies, it was further discarded that anti-TFAM antibodies in SLE were cross-reactive with β2GPI (Supplemental FIG. 2B). Since patients with PAPS are defined by the presence of APS-associated antibodies, it was not surprising that anti-TFAM antibodies were not associated with any particular antibody in patients with PAPS (Table 4A).

TABLE 4A
Associations between Anti-TFAM antibodies and APS-
associated antibodies in patients with PAPS.

Anti-TFAM

| | Negative | | Positive | | p |
|---|---|---|---|---|---|
| | 42 (84%) | | 8 (16%) | | value |
| Anti-β2GPI IgG, n (%) | 17 | (40) | 2 | (25) | 0.45 |
| Anti- β2GPI IgM, n (%) | 7 | (17) | 2 | (25) | 0.65 |
| ACL IgG, n (%) | 13 | (31) | 3 | (37) | 1 |
| ACL IgM, n (%) | 10 | (25) | 1 | (12.5) | 0.66 |
| LAC, n (%) | 33 | (78) | 7 | (87.5) | 1 |

B2GPI, anti-β2-glycoprotein-I antibodies. ACL, anticardiolipin. LAC, lupus anticoagulant. Associations between frequencies were determines using Fisher's exact test.

### Anti-TFAM Antibodies are Predictive of Thrombotic Events in SLE

To analyze the predictive value of anti-TFAM antibodies and possible covariates, SLE patients were classified in two groups: 1) subjects with any thrombotic event (n=54, 34%), and 2) subjects without thrombosis (n=102, 65.4%) (Table 4B).

TABLE 4B
Clinical and laboratory associations present during the clinical
course of patients with SLE who had any thrombotic event.

| | Any thrombotic event | | | | |
|---|---|---|---|---|---|
| | Absent | | Present | | p. |
| Clinical | n = 102 (100%) | | n = 54 (100%) | | value |
| *Age, yrs | 49.4 | (38.5-60.0) | 52.3 | (43.2-57.6) | 0.239 |
| *Disease Duration, | 17.9 | (10-23) | 23.8 | (14.2-32.2) | 0.003 |
| yrs | | | | | |
| Sex | 6 | (5.9) | 1 | (1.9) | 0.423 |
| Black | 40 | (88.9) | 52 | (91.2) | 1 |
| CVD risk factors | | | | | |
| Smoking past | 27 | (26.5) | 32 | (59.3) | <0.001 |
| Alcoholism past | 4 | (3.9) | 7 | (13) | 0.049 |
| Hypertension | 77 | (75.5) | 39 | (72.2) | 0.702 |
| Obesity | 58 | (56.9) | 36 | (66.7) | 0.302 |
| Cholesterol | 67 | (65.7) | 39 | (72.2) | 0.473 |
| Triglycerides | 25 | (24.5) | 15 | (27.8) | 0.702 |
| Diabetes | 14 | (13.7) | 12 | (22.2) | 0.183 |
| Constitutional | | | | | |
| Fever | 32 | (31.4) | 23 | (42.6) | 0.217 |
| Renal | | | | | |
| Proteinuria | 46 | (45.1) | 28 | (51.9) | 0.501 |
| Nephrotic syndrome | 19 | (18.6) | 5 | (9.3) | 0.163 |
| Hematuria | 31 | (30.4) | 18 | (33.3) | 0.72 |
| Renal Insufficiency | 20 | (19.6) | 16 | (29.6) | 0.168 |
| Renal Failure | 2 | (2) | 5 | (9.3) | 0.049 |
| Cutaneous | | | | | |
| Malar rash | 55 | (53.9) | 35 | (64.8) | 0.234 |
| Discoid | 21 | (20.6) | 12 | (22.2) | 0.838 |
| Photosensitivity | 49 | (48) | 39 | (72.2) | 0.004 |
| Mouth ulcers | 56 | (54.9) | 34 | (63) | 0.395 |
| Alopecia | 64 | (62.7) | 38 | (70.4) | 0.38 |
| Raynaud | 54 | (52.9) | 36 | (66.7) | 0.125 |
| SCLE | 7 | (6.9) | 2 | (3.7) | 0.72 |
| Vasculitis | 11 | (10.8) | 10 | (18.5) | 0.219 |
| Leg ulcers | 0 | (0) | 4 | (7.4) | 0.013 |
| Panniculitis | 2 | (2) | 3 | (5.6) | 0.342 |
| Livedo | 32 | (31.4) | 24 | (44.4) | 0.117 |
| Musculoskeletal | | | | | |
| Arthralgia | 95 | (93.1) | 51 | (94.4) | 1 |
| Arthritis | 79 | (77.5) | 45 | (83.3) | 0.414 |
| Erosions | 2 | (2.9) | 0 | (0) | 0.536 |
| Myositis | 12 | (11.8) | 4 | (7.4) | 0.58 |
| Serositis | | | | | |
| Pleuritis | 53 | (52) | 33 | (61.1) | 0.312 |
| Pericarditis | 25 | (24.8) | 17 | (31.5) | 0.448 |
| Neurological | | | | | |
| Seizure | 4 | (3.9) | 4 | (7.4) | 0.449 |
| Psychosis | 2 | (2) | 1 | (1.9) | 1 |
| Organic brain | 4 | (3.9) | 5 | (9.3) | 0.277 |
| syndrome | | | | | |
| Meningitis | 0 | (0) | 2 | (3.7) | 0.118 |
| Stroke | 0 | (0) | 7 | (13) | <0.001 |
| Depression | 39 | (38.2) | 23 | (42.6) | 0.61 |
| Headache | 6 | (5.9) | 9 | (16.7) | 0.044 |
| Mononeuritis | 0 | (0) | 2 | (3.7) | 0.118 |
| multiplex | | | | | |
| Cognitive | 8 | (7.8) | 6 | (11.1) | 0.56 |
| impairment | | | | | |
| Optic neuritis | 0 | (0) | 0 | (0) | 1 |
| Cranial neuropathy | 1 | (1) | 4 | (7.4) | 0.049 |
| Peripheral | 5 | (4.9) | 3 | (5.6) | 1 |
| neuropathy | | | | | |
| Transverse myelitis | 3 | (2.9) | 1 | (1.9) | 1 |
| Hematological | | | | | |
| Lymphadenopathy | 37 | (36.3) | 28 | (51.9) | 0.087 |
| Anemia | 77 | (75.5) | 39 | (72.2) | 0.702 |
| Hemolytic anemia | 6 | (5.9) | 4 | (7.4) | 0.739 |
| Coombs test | 12 | (11.8) | 13 | (24.1) | 0.065 |
| Leukopenia | 46 | (45.1) | 22 | (40.7) | 0.616 |
| Lymphopenia | 47 | (46.1) | 20 | (37) | 0.311 |
| Platelet | 24 | (23.5) | 17 | (31.5) | 0.34 |
| Lupus anticoagulant | 28 | (27.5) | 26 | (48.1) | 0.013 |
| Cardiac | | | | | |
| Myocarditis | 2 | (2) | 1 | (1.9) | 1 |
| Libman-Sacks | 2 | (2) | 1 | (1.9) | 1 |
| Murmur | 52 | (51) | 33 | (61.1) | 0.242 |
| Pulmonary | | | | | |
| Lung fibrosis | 17 | (16.7) | 6 | (11.1) | 0.478 |
| Pulmonary | 16 | (15.7) | 6 | (11.1) | 0.48 |
| hypertension | | | | | |
| Gastrointestinal, | | | | | |
| GI lupus | 11 | (10.8) | 3 | (5.6) | 0.382 |
| Hepatomegaly | 5 | (4.9) | 0 | (0) | 0.164 |
| Abnormal liver | 50 | (49) | 22 | (40.7) | 0.399 |
| function tests | | | | | |
| Splenomegaly | 1 | (1) | 1 | (1.9) | 1 |
| Pancreatitis | 2 | (2) | 3 | (5.6) | 0.342 |
| Sjogren Syndrome | 28 | (27.4) | 16 | (29.6) | 0.852 |
| Immunological | | | | | |
| Anti-DNA | 62 | (60.8) | 36 | (66.7) | 0.492 |
| Anti-Sm | 20 | (19.6) | 11 | (20.4) | 1 |
| Ro52ex4 | 27 | (26.5) | 20 | (37) | 0.201 |
| Anti-La | 15 | (14.7) | 9 | (16.7) | 0.817 |
| Anti-RNP | 28 | (27.5) | 13 | (24.1) | 0.705 |
| Anticardiolipin | 66 | (64.7) | 36 | (66.7) | 0.861 |
| Anti-B2GPI | 31 | (30.7) | 17 | (32.1) | 0.857 |
| Anti-TFAM | 23 | (22.5) | 25 | (46.3) | 0.003 |
| FPRPR | 9 | (8.8) | 9 | (16.7) | 0.188 |
| CH50 | 14 | (13.7) | 9 | (16.7) | 0.64 |
| Low C3 | 53 | (52) | 32 | (59.3) | 0.403 |
| Low C4 | 42 | (41.2) | 30 | (55.6) | 0.094 |
| ESR | 74 | (72.5) | 43 | (79.6) | 0.437 |
| Thrombotic, n (%) | | | | | |
| Transient ischemic | 0 | (0) | 5 | (9.3) | 0.004 |
| attack | | | | | |
| Superficial | 0 | (0) | 9 | (16.7) | <0.001 |
| thrombosis | | | | | |
| Deep vein | 0 | (0) | 27 | (50) | <0.001 |
| thrombosis | | | | | |
| Cerebrovascular | 0 | (0) | 21 | (38.9) | <0.001 |
| accident | | | | | |
| Myocardial | 0 | (0) | 3 | (5.6) | 0.04 |
| infarction | | | | | |
| Digital thrombosis | 0 | (0) | 6 | (11.1) | 0.001 |
| Other venous | 0 | (0) | 5 | (9.3) | 0.004 |
| thrombosis | | | | | |
| Venous thrombosis | 0 | (0) | 33 | (61.1) | <0.001 |
| Arterial thrombosis | 0 | (0) | 31 | (57.4) | <0.001 |
| Other arterial | 0 | (0) | 6 | (11.1) | 0.001 |
| thrombosis | | | | | |
| Avascular necrosis | 11 | (10.8) | 12 | (22.2) | 0.062 |
| APS | 3 | (2.9) | 43 | (79.6) | <0.001 |
| Preeclampsia | 5 | (4.9) | 3 | (5.5) | 1 |
| Miscarriages | 19 | (18.6) | 16 | (29.6) | 0.157 |

All variables are summarized as n (%) unless otherwise indicated. Associations between binary variables were estimated using Fisher's exact test.
*These variables were summarized as mean (IQR), P values were estimated using Student's T test. SCLE, Subacute cutaneous lupus erythematosus. Anti-B2GPI, Anti-β2 Glycoprotein I. FP-RPR, false positive rapid plasma regain test. ESR, erythrocyte sedimentation rate.

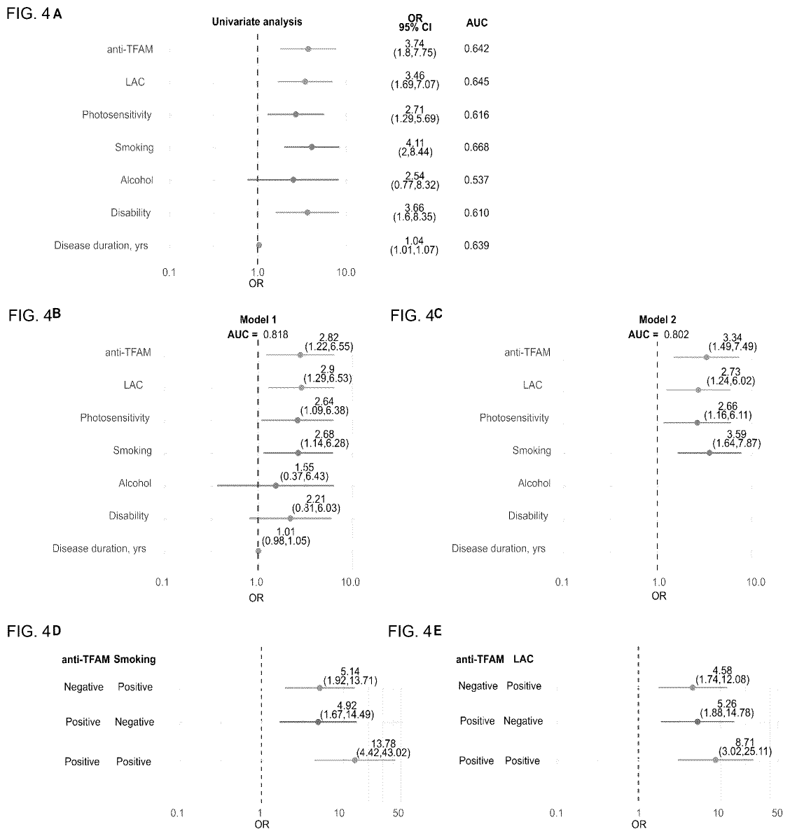

Univariate analysis revealed that patients who had thrombotic events had longer disease duration (23.7 vs. 18.5 years, OR=1.04 per year of disease duration, P=0.01) (FIG. 4A). Notably, only anti-TFAM antibodies and LAC (determined by increased dilute Rusell viper venom test, dRVVT) were significantly associated with any thrombotic event [46% (25/54) vs. 23% (23/102), OR=3.74, p=0.003; and 50% (27/54) vs. 27% (28/102), OR=3.46, P=0.007] (FIG. 4A and Table 4). Similar to other studies in SLE (Galli et al., 2003; Previtali et al., 2002), however, anti-β2GPI or anti-cardiolipin (ACL) antibodies were not associated with thrombotic events in the SPARE cohort (Table 4B) (Galli et al., 2003; Previtali et al., 2002).

Among traditional cardiovascular risk factors, prior smoking was significantly associated with thrombotic events [59% (32/54) vs. 26.5% (27/102), OR=4.1, p<0.001] (FIG. 4A and Table 4B), and among the clinical manifestations not known to be related to thromobosis, photosensitivity was more frequent in patients with a history of thrombosis [72.2% (39/54) vs. 48% (49/102), OR=2.71, p=0.004] (FIG. 4A and Table 4B). Patients with thrombosis were also more likely to have some degree of disability (OR=3.66) (FIG. 4A). Patients with thrombosis were also more likely to have some degree of disability (OR=3.66). After adjustment for LAC, photosensitivity, and smoking, SLE patients positive for anti-TFAM antibodies were 2.82 to 3.34 times more likely to have a thrombotic event during their clinical course than anti-TFAM negative patients (FIG. 4B and FIG. 4C). Interestingly, whereas the likelihood of having a thrombotic event was similar among patients positive for anti-TFAM antibodies or having a smoking history (OR=4.92 and 5.14, respectively), an additive effect was found when both conditions were present (OR=13.78) (FIG. 4D). Similarly, the presence of LAC had an additive effect on the risk of thrombosis in SLE patients positive for anti-TFAM antibodies (OR=8.71) (FIG. 4E).

### Antibodies to TFAM are Associated to Transcriptional Fingerprints Linked to Thrombosis in SLE.

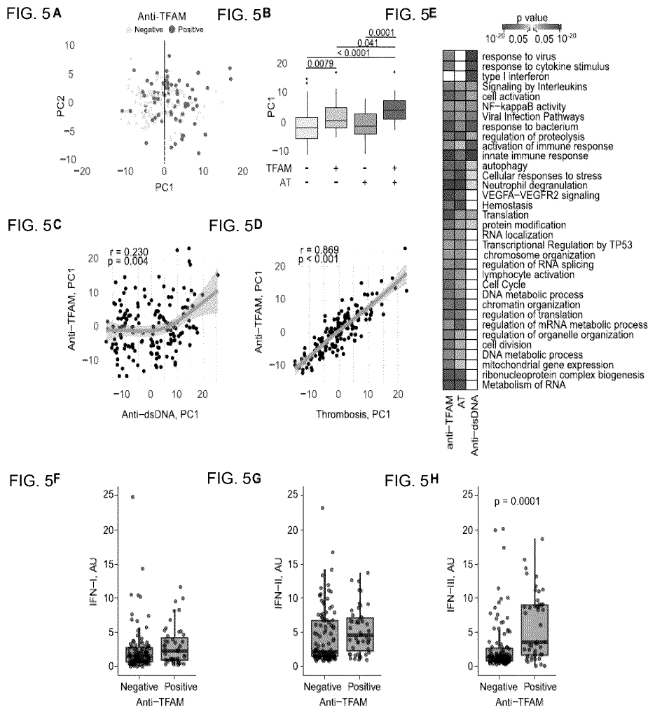

Since the extracellular released of nucleoids from activated neutrophils has been associated with the induction of IFN-I in SLE (Bennett et al., 2003), it was addressed whether anti-TFAM antibodies are linked to unique transcriptional profiles-particularly the IFN signature-using gene expression data from blood collected in parallel with the samples used to measure anti-TFAM antibodies. To uncover gene expression signatures associated to anti-TFAM antibodies, principal components analysis (PCA) was used on the top 500 transcripts ranked by their Fisher score between SLE patients positive and negative for anti-TFAM antibodies (i.e., the anti-TFAM transcriptome) (Table 5). The first principal component (PC1) stratified SLE patients on a gradient rather than specific clusters (FIG. 5A). PC1 was significantly higher in SLE patients who were positive for anti-TFAM antibodies and had a history of thrombotic events (FIG. 5B).

TABLE 5
Top 500 Transcripts ranked according to the Fisher's score
in SLE Patients Positive vs. Negative for Anti-TFAM Antibodies

| Gene | Fisher Score |
|---|---|
| SLC35A2 | 0.117960478 |
| GPRASP1 | 0.116869331 |
| RYK | 0.115473218 |
| EIF3E | 0.112515878 |
| ZNF439 | 0.104267975 |
| TOB2 | 0.09918545 |
| SAPS3 | 0.099151432 |
| ZFP36L2 | 0.098584465 |
| PDP1 | 0.09609193 |
| CAND2 | 0.095664324 |
| ASNSD1 | 0.094451169 |
| WDR45L | 0.093841147 |
| HLA-DPB1 | 0.093404269 |
| DPP8 | 0.093034572 |
| SESN1 | 0.09064832 |
| CSRP1 | 0.089410113 |
| KIAA1432 | 0.089210392 |
| NOG | 0.086292259 |
| TMEM194A | 0.083084068 |
| H2AFJ | 0.082684045 |
| ARMC1 | 0.08252909 |
| SECISBP2L | 0.082022839 |
| PAQR7 | 0.081927126 |
| AMY2B /// RNPC3 | 0.081851785 |
| RBM6 | 0.081593614 |
| ZNF252 | 0.08123122 |
| FLVCR1 | 0.079629633 |
| ARID1B | 0.07940298 |
| BLCAP | 0.078915758 |
| HNRNPA3 | 0.078740381 |
| PID1 | 0.078237057 |
| C12orf26 | 0.077886815 |
| SETD6 | 0.077158973 |
| TBC1D9 | 0.076449498 |
| FBXW7 | 0.07623258 |
| USP9X | 0.076161399 |
| ALDH1A1 | 0.07514022 |
| AEN | 0.073771151 |
| FNTA | 0.073242578 |
| CTR9 | 0.073197031 |
| TSPYL1 | 0.072081971 |
| ANKS6 | 0.071962053 |
| LOC254128 | 0.071492265 |
| KRR1 | 0.070395315 |
| EP400 | 0.069766625 |
| BTG1 | 0.069582295 |
| EPPK1 | 0.069282654 |
| RPL23 /// SNORA21 | 0.068840508 |
| ZSCAN2 | 0.068594402 |
| LPCAT1 | 0.068144047 |
| ADRBK2 | 0.067533096 |
| ZNF236 | 0.067413604 |
| BRD1 | 0.066848189 |
| PDE4B | 0.066603617 |
| OGFRL1 | 0.066535708 |
| HP1BP3 | 0.066060034 |
| SLC7A6OS | 0.065951937 |
| SGK1 | 0.065813958 |
| PICALM | 0.065434067 |
| ATM | 0.065043615 |
| LOC158257 | 0.065014761 |
| TMEM65 | 0.063976066 |
| SBDS /// SBDSP1 | 0.063958176 |
| C1orf63 | 0.06333914 |
| MST4 | 0.06240872 |
| KIAA0355 | 0.061827222 |
| LOC100288939 | 0.061783927 |
| LFNG | 0.061621904 |
| NHS | 0.0615563 |
| SETD7 | 0.061222237 |
| ING3 | 0.060248392 |
| TOPORS | 0.059943044 |
| SRRM2 | 0.059935752 |
| AKAP1 | 0.05967078 |
| TRIT1 | 0.059463786 |
| HERC4 | 0.059398645 |
| MT1H /// MT1P2 | 0.059339913 |
| PTPRS | 0.058939212 |
| LMLN | 0.058584831 |
| UBR3 | 0.058247341 |
| STRBP | 0.058049741 |
| MED6 | 0.057788891 |
| MT1F | 0.057753717 |
| PTGER4 | 0.05767236 |
| ZNF550 | 0.057476086 |
| QSOX1 | 0.057207157 |
| SLTM | 0.057146035 |
| RNF44 | 0.056884537 |
| NAA38 | 0.056792132 |
| GALNT7 | 0.056501373 |
| CS | 0.056439272 |
| LILRA4 | 0.05620712 |
| ISG20L2 | 0.056113303 |
| MAPK8 | 0.05609904 |
| C13orf18 | 0.056038117 |
| MAT2B | 0.055992937 |
| CDKN1A | 0.055582403 |
| PGM2L1 | 0.055436028 |
| ATP9A | 0.055178942 |
| ETFB | 0.055147085 |
| LOC283588 | 0.055072469 |
| ZNF302 | 0.054584563 |
| SUMF1 | 0.054531252 |
| TTC3 | 0.054390256 |
| RAPH1 | 0.054389198 |
| TERF1 | 0.054186965 |
| RTN1 | 0.054079139 |
| MTMR1 | 0.054063711 |
| NAAA | 0.053914785 |
| RAD21 | 0.053894966 |
| BRI3BP | 0.05388177 |
| SPTY2D1 | 0.053629299 |
| FBXO41 | 0.053612359 |
| MFSD8 | 0.053572993 |
| GM2A | 0.053411552 |
| EEF1B2 | 0.053342449 |
| TATDN3 | 0.053266014 |
| CYP4V2 | 0.052936972 |
| GLG1 | 0.052791521 |
| C9orf156 | 0.052758629 |
| NCRNA00204 | 0.052753056 |
| TRERF1 | 0.052493561 |
| SFRS18 | 0.052417528 |
| CCDC47 | 0.05238699 |
| C14orf43 | 0.052182814 |
| PTP4A2 | 0.05207546 |
| CPVL | 0.05206048 |
| GRLF1 | 0.051989282 |
| C19orf59 | 0.051738237 |
| FAM117B | 0.051713564 |
| C1orf77 | 0.051686994 |
| GPR183 | 0.051672697 |
| SNX30 | 0.05166893 |
| EEF1G /// TUT1 | 0.051641637 |
| VMA21 | 0.051614728 |
| FRMD8 | 0.051410896 |
| H2BFS | 0.05134876 |
| PPTC7 | 0.051276438 |
| RPS6KA5 | 0.051207996 |
| ZNF281 | 0.051192207 |
| EEF1A1 /// EEF1A1P24 /// EEF1A1P9 | 0.050954318 |
| SMARCD3 | 0.050947468 |
| PRPF38B | 0.050892871 |
| AFMID /// IDS | 0.050350748 |
| GOLGA4 | 0.050022603 |
| BCL11B | 0.049968305 |
| TDG | 0.04991504 |
| DHX9 | 0.049871043 |
| MT1P2 | 0.049821403 |
| NOL9 | 0.049819694 |
| DHX58 | 0.049652015 |
| WHSC2 | 0.049613304 |
| LOC339290 | 0.049571003 |
| TPRKB | 0.049400555 |
| TSC22D2 | 0.049336252 |
| NACC2 | 0.049325328 |
| PHC3 | 0.049181328 |
| ORMDL1 | 0.049096395 |
| LOC400960 | 0.049077264 |
| FCRL3 | 0.048982149 |
| TMEM168 | 0.048876776 |
| C11orf30 | 0.048743909 |
| LGALS3BP | 0.048710847 |
| LGALS1 | 0.048625416 |
| C7orf60 | 0.048477814 |
| CREBZF | 0.04834792 |
| SFRS2 | 0.048346582 |
| ZNF238 | 0.04818724 |
| LOC100170939 | 0.048175743 |
| EIF4A3 | 0.048138925 |
| EIF2S3 | 0.048053121 |
| CAND1 | 0.048026778 |
| NAPG | 0.047977813 |
| ZNF117 | 0.047674593 |
| DYRK1A | 0.047513656 |
| SET | 0.047331488 |
| FOXO1 | 0.046914538 |
| FGL2 | 0.046804517 |
| KIAA0415 | 0.046738909 |
| TET3 | 0.046629255 |
| ZNF468 | 0.046535095 |
| S100A9 | 0.046505129 |
| ATRX | 0.04628328 |
| SMC5 | 0.046267737 |
| GABPA | 0.04597441 |
| ALG6 | 0.04568656 |
| MDN1 | 0.045676292 |
| HLA-DQA1 /// HLA-DQA2 | 0.045675115 |
| C10orf12 | 0.045604824 |
| HIST1H2BE | 0.045557675 |
| CSDE1 | 0.045540736 |
| PDPK1 | 0.045521208 |
| MLF1IP | 0.045518391 |
| KIAA0907 | 0.0455044 |
| ZNF652 | 0.045454183 |
| UPP1 | 0.045339099 |
| SUN1 | 0.04513395 |
| RPL35A | 0.045133662 |
| BTBD7 | 0.045112549 |
| FAM122B | 0.045047185 |
| C1orf9 | 0.04495854 |
| ITSN2 | 0.044947512 |
| SEC11A | 0.044918578 |
| LIX1L | 0.044860988 |
| OGDH | 0.044763461 |
| PTCRA | 0.044714363 |
| C1orf174 | 0.044666936 |
| HIST1H2AJ | 0.044643777 |
| RANBP2 | 0.044574607 |
| NBR1 | 0.044507976 |
| SNRPB2 | 0.044493816 |
| UBE4A | 0.044488203 |
| DAPK1 | 0.04441643 |
| CACNA2D3 | 0.044412342 |
| FCRL1 | 0.044359209 |
| FXC1 | 0.044247154 |
| NPAT | 0.044167338 |
| HECA | 0.044124721 |
| GALK1 | 0.044050754 |
| AFF3 | 0.043906627 |
| OTUD4 | 0.043827201 |
| POLR2B | 0.043813941 |
| CAMK2D | 0.043794144 |
| POLR2C | 0.043766979 |
| KIAA1737 | 0.043626452 |
| GGNBP2 | 0.043566818 |
| SLC7A6 | 0.043529394 |
| ZNF770 | 0.043452619 |
| TOMM20 | 0.043401939 |
| USP22 | 0.04338131 |
| MPEG1 | 0.043268564 |
| SERF1A /// SERF1B | 0.043198862 |
| ACACB | 0.043136685 |
| OLFM4 | 0.043065649 |
| RWDD4A | 0.043047088 |
| EIF5 | 0.042988953 |
| AHSP | 0.042911758 |
| RPA2 | 0.042785957 |
| FAM108B1 | 0.042749742 |
| PPP3CA | 0.042748154 |
| HLA-DMB | 0.042703801 |
| CDKN1B | 0.042549009 |
| HK3 | 0.042526204 |
| SFRS5 | 0.042503805 |
| TMEM179B | 0.042501145 |
| REST | 0.0424121 |
| LIN7C | 0.042368948 |
| UFM1 | 0.042359784 |
| WDR89 | 0.042359373 |
| FCRL2 | 0.042342125 |
| KIAA0495 | 0.042310035 |
| CD46 | 0.042296809 |
| IRF8 | 0.042290993 |
| ZYG11B | 0.04226207 |
| CCR6 | 0.042260917 |
| ZBTB43 | 0.042180035 |
| GRINA | 0.042090629 |
| FCRLA | 0.042060897 |
| ERCC1 | 0.042012653 |
| C12orf32 | 0.042005343 |
| DYNLT3 | 0.04180435 |
| BAG5 | 0.041707321 |
| FUBP3 | 0.041675123 |
| LOC100288730 | 0.041647283 |
| UBE2E1 | 0.041554849 |
| SH2B3 | 0.041533441 |
| LOC149832 | 0.041489728 |
| MEAF6 | 0.041441631 |
| TCEB3 | 0.041383942 |
| TBC1D5 | 0.041295263 |
| NPM1 | 0.041280937 |
| LUZP6 /// MTPN | 0.041272641 |
| SACS | 0.041176849 |
| BDP1 | 0.04117652 |
| GPATCH8 | 0.04116939 |
| SART1 | 0.041167359 |
| DIS3L | 0.041163438 |
| ACTR10 | 0.041158599 |
| GOLGA7 | 0.041146369 |
| TPR | 0.041114568 |
| MGAT4A | 0.041015342 |
| TRAK1 | 0.040996778 |
| HIPK2 | 0.040976319 |
| CBFA2T2 | 0.040959117 |
| UBP1 | 0.04080237 |
| SIAH1 | 0.040781453 |
| MAPK1 | 0.040769788 |
| EIF4B | 0.040704121 |
| MMP8 | 0.040685787 |
| LOC100271836 /// LOC440354 /// | 0.04061785 |
| LOC595101 /// LOC641298 /// SMG1 | |
| ZNF518B | 0.040606496 |
| GVIN1 | 0.040504841 |
| IL24 | 0.040404251 |
| RORA | 0.040398043 |
| CDK2AP1 | 0.040354917 |
| TAF11 | 0.040275053 |
| ASXL2 | 0.040271238 |
| C10orf137 | 0.040262423 |
| BANK1 | 0.040260071 |
| UTS2 | 0.040226886 |
| MRPS25 | 0.040092736 |
| GBA /// GBAP1 | 0.040071185 |
| OGT | 0.040045785 |
| PCMTD2 | 0.039984354 |
| SOCS7 | 0.03989967 |
| INPP5F | 0.039898555 |
| ENSA | 0.039716973 |
| LDOC1L | 0.039698289 |
| PKN2 | 0.039561591 |
| THAP4 | 0.039521105 |
| ZNF395 | 0.039515426 |
| TMCC2 | 0.039430497 |
| KIAA0753 | 0.039394616 |
| WDR82 | 0.039324124 |
| KLHL36 | 0.039167962 |
| EIF2A | 0.039094334 |
| WDR73 | 0.039082131 |
| TM9SF2 | 0.039035983 |
| ATP6V1B2 | 0.03903205 |
| ZNF217 | 0.038964012 |
| CYB561D1 | 0.03895956 |
| HLA-DRB1 | 0.038926686 |
| BUD13 | 0.038790732 |
| ZNF207 | 0.038753769 |
| RFX5 | 0.038749537 |
| DDX26B | 0.038727597 |
| ABLIM1 | 0.038709326 |
| RPS4X | 0.038665479 |
| BUB3 | 0.038484227 |
| SAR1B | 0.038468393 |
| TES | 0.038467875 |
| PPME1 | 0.038430596 |
| COQ10B | 0.038376347 |
| NOL12 /// TRIOBP | 0.038371393 |
| TNFRSF10B | 0.038303901 |
| ZBTB40 | 0.03829665 |
| PREPL | 0.038295368 |
| BMI1 | 0.038282275 |
| SYNJ1 | 0.038150297 |
| ZNF260 | 0.038058699 |
| STK11 | 0.037999496 |
| LRIG1 | 0.037979165 |
| DDX1 | 0.037974598 |
| SYPL1 | 0.037909522 |
| HBP1 | 0.037892929 |
| ZNF862 | 0.037887277 |
| LRPPRC | 0.037662138 |
| MPZL1 | 0.037645746 |
| CHP | 0.037512381 |
| SENP6 | 0.037422937 |
| FAM129C | 0.037244379 |
| HNRNPH1 | 0.037129072 |
| TARDBP | 0.037087354 |
| COG2 | 0.037000666 |
| ASXL1 | 0.036929073 |
| IFI27 | 0.036908073 |
| SCAI | 0.036907923 |
| FOXK1 | 0.036855628 |
| NFXL1 | 0.036797822 |
| SERINC3 | 0.036714221 |
| LOC283663 | 0.03668575 |
| TRAF5 | 0.036536067 |
| YY1 | 0.036505899 |
| MRPS27 | 0.036459601 |
| XPNPEP3 | 0.036404841 |
| ZFP36L1 | 0.036376669 |
| LOC96610 | 0.03629287 |
| PSMC6 | 0.036283243 |
| CGGBP1 | 0.036272253 |
| RAB32 | 0.036135987 |
| ZC3H14 | 0.03612773 |
| FRY | 0.03610339 |
| TSPAN13 | 0.036094896 |
| ZNF418 | 0.036043911 |
| TNKS | 0.036001122 |
| AMPD2 | 0.035994816 |
| NCBP2 | 0.035924952 |
| TOP2B | 0.035911111 |
| ATP8A1 | 0.035864174 |
| RBM25 | 0.035817588 |
| C16orf52 | 0.035803494 |
| TSHZ1 | 0.035785454 |
| EPB41L2 | 0.035734704 |
| SIGLEC7 | 0.03569199 |
| TAX1BP1 | 0.035671862 |
| HELZ | 0.035671775 |
| C20orf27 | 0.035647611 |
| TNRC6B | 0.035624651 |
| WRB | 0.035612965 |
| HMGN3 | 0.035604596 |
| LRMP | 0.035594223 |
| FAM174A | 0.035586772 |
| FCER1A | 0.035545936 |
| F11R | 0.035511415 |
| GRPEL2 | 0.035503194 |
| ARPC5 | 0.03546303 |
| ELK4 | 0.035409805 |
| ZNF107 | 0.035370702 |
| MRI1 | 0.035342882 |
| KCTD12 | 0.03531621 |
| ARMCX3 | 0.035276272 |
| DUSP28 | 0.035255932 |
| EIF3A | 0.035253024 |
| KIAA1797 | 0.035219955 |
| MT1G | 0.035216217 |
| ZNF182 | 0.0351997 |
| RBM39 | 0.035191583 |
| FAM153A /// FAM153B | 0.03514584 |
| KIAA2026 | 0.035104873 |
| FANCD2 | 0.035100493 |
| UBN2 | 0.035089637 |
| CCDC159 | 0.035069332 |
| BUD31 | 0.035027354 |
| C6orf204 | 0.035026944 |
| HSF2 | 0.035021579 |
| ACRBP | 0.035015526 |
| ZNF548 | 0.034996062 |
| MT1X | 0.034967801 |
| WDR44 | 0.034954903 |
| PITPNB | 0.034931814 |
| AUTS2 | 0.03492383 |
| PLP2 | 0.03480321 |
| QSOX2 | 0.034787822 |
| DCAF17 | 0.034765009 |
| TYMP | 0.034762034 |
| ANKRD46 | 0.034757933 |
| FAM102A | 0.034757888 |
| MSI2 | 0.034719334 |
| LOC643008 | 0.034588701 |
| GPR68 | 0.0345819 |
| CDC42 | 0.034566855 |
| POP5 | 0.034561231 |
| OAZ2 | 0.034554515 |
| IGF1R | 0.034501578 |
| USP34 | 0.03446327 |
| AFTPH | 0.034459234 |
| SEL1L | 0.034458398 |
| C19orf50 | 0.034434354 |
| ATXN1L | 0.034417887 |
| BCAP29 | 0.034416316 |
| CAPRIN2 | 0.034411326 |
| STK38 | 0.034406905 |
| DUSP7 | 0.034399718 |
| FAM100B | 0.034384467 |
| RLIM | 0.034366688 |
| HLA-DRA | 0.034244326 |
| STX16 | 0.034243872 |
| KSR1 | 0.034194825 |
| SF3B4 | 0.034194286 |
| CAPZA2 | 0.034177495 |
| PTPRE | 0.034153444 |
| SETD5 | 0.034146087 |
| CCDC14 | 0.034110135 |
| ANKH | 0.034087434 |
| DPY19L1 | 0.034009657 |
| ZNF655 | 0.034009601 |
| USP8 | 0.033992528 |
| ZNF12 | 0.033987065 |
| CLASP2 | 0.033962104 |
| SEC63 | 0.033943011 |
| TMEM206 | 0.033920483 |
| GNL1 | 0.033911361 |
| SAMD8 | 0.033886862 |
| CTSS | 0.03386717 |
| DYRK2 | 0.03385669 |
| ATP6AP2 | 0.03381541 |
| PRDM2 | 0.033784505 |
| CSTF3 | 0.033750115 |
| RCAN3 | 0.033658769 |
| ALKBH5 | 0.033630115 |
| EIF1 | 0.03359264 |
| LOC221442 | 0.033583553 |
| ZNF706 | 0.033581188 |
| TSC1 | 0.033555333 |
| MGA | 0.033471335 |
| IPO7 | 0.033456706 |
| SVIP | 0.033449192 |
| ST3GAL2 | 0.033444238 |
| KDM2B | 0.033442173 |
| C3orf23 | 0.033400141 |
| C11orf68 | 0.0333757 |
| DYNC1I2 | 0.033345276 |
| ZMYND11 | 0.033340837 |
| ZNF844 | 0.033333527 |
| CPPED1 | 0.033291256 |
| H2AFZ | 0.033284384 |
| AKAP9 | 0.033173814 |
| RPL7L1 | 0.033141675 |
| ZFAND5 | 0.033114065 |
| CCDC117 | 0.033096718 |
| SNRPC | 0.033077685 |
| ALDH9A1 | 0.033066605 |
| ZNF248 | 0.033035228 |
| GNG5 | 0.033034033 |
| DPYSL2 | 0.033003444 |
| TMEM203 | 0.033001179 |
| SMARCE1 | 0.032998614 |
| BNIP3 | 0.03299568 |
| MGC16275 | 0.032951558 |
| LOC374443 | 0.032942542 |

To compare the transcriptome of anti-TFAM antibodies to those associated with thrombosis or activation by anti-dsDNA antibodies, PCA analysis was performed using the top 500 transcripts ranked by their Fisher score between patients with and without history of thrombosis (i.e., thrombosis transcriptome; Table 6) and between patients positive and negative for anti-dsDNA antibodies (i.e., anti-dsDNA transcriptome; Table 7), respectively. Interestingly, while anti-TFAM antibody PC1 showed only mild correlation with anti-dsDNA antibody PC1 (r=0.230, P=0.004), it had a strong correlation with thrombosis PC1 (r=0.869, p=0.001) (FIG. 5C and FIG. 5D, respectively). There was no correlation between anti-dsDNA antibody PC1 and thrombosis (r=0.01, P=0.914). Together, these data imply that PC1 represents transcripts and pathways associated with anti-TFAM-related thrombosis rather than anti-dsDNA mediated immune activation.

TABLE 6
Top 500 Transcripts ranked according to the Fisher's
score in Patients with and without History of Thrombosis

| Gene | Fisher Score |
|---|---|
| TRA@ /// TRD@ | 0.098387859 |
| DULLARD | 0.08433828 |
| THTPA | 0.082769292 |
| SIK3 | 0.081347481 |
| NUCB1 | 0.080396843 |
| TCF7L2 | 0.079592381 |
| NOTCH2NL | 0.072909204 |
| HNRNPA3 | 0.072407693 |
| ZNF652 | 0.071211027 |
| EIF3B | 0.069363601 |
| TRAK1 | 0.067161248 |
| C5orf56 | 0.065583143 |
| YTHDC1 | 0.064592618 |
| SFRS14 | 0.064077418 |
| FGD2 | 0.063697802 |
| DENND4B | 0.062196607 |
| FGL2 | 0.061895743 |
| DLG5 | 0.059148283 |
| LOC100131541 | 0.058849188 |
| FLT1 | 0.05805784 |
| LOC200772 | 0.056645027 |
| S1PR5 | 0.056309685 |
| KIAA1245 /// LOC100288142 /// | 0.05592551 |
| LOC200030 /// NBPF1 /// NBPF10 /// | |
| NBPF11 /// NBPF14 /// NBPF15 /// | |
| NBPF16 /// NBPF8 /// NBPF9 | |
| WDR13 | 0.054964132 |
| ZNF439 | 0.054826762 |
| KIAA1429 | 0.054057044 |
| PTPRE | 0.05405692 |
| REL | 0.053518099 |
| AHSA2 | 0.05296 |
| PHF20 | 0.052546126 |
| BRD8 | 0.052231796 |
| PPP2CA | 0.052176731 |
| MLF2 | 0.051805193 |
| DNAJC13 | 0.05100804 |
| LOC158257 | 0.05071987 |
| CDCA7L | 0.050673418 |
| LOC339290 | 0.050399217 |
| PRPF3 | 0.050380696 |
| C9orf91 | 0.050283563 |
| CTNND1 | 0.050160934 |
| CCNL1 | 0.04983434 |
| FLVCR1 | 0.048336129 |
| DYRK1A | 0.047981772 |
| RBM6 | 0.047810723 |
| TIMP1 | 0.04764439 |
| METTL9 | 0.047571788 |
| LSM2 | 0.047566377 |
| KLRB1 | 0.047139211 |
| KIAA0922 | 0.04710864 |
| TBC1D22B | 0.047032444 |
| ZFAND5 | 0.047003243 |
| NLRP1 | 0.04675978 |
| MIR101-1 | 0.046562319 |
| BSG | 0.046556614 |
| IP6K2 | 0.046307573 |
| PMS2L11 | 0.046239059 |
| LOC100128510 | 0.045989092 |
| SFRS18 | 0.045918496 |
| LILRA2 | 0.045714333 |
| SIAH2 | 0.045538521 |
| PLP2 | 0.045085557 |
| RPGRIP1 | 0.044949834 |
| NDUFV3 | 0.04448485 |
| CHMP4B | 0.044249965 |
| YPEL1 | 0.044171607 |
| HADH | 0.043983684 |
| ZFP36L2 | 0.043726339 |
| UBE4A | 0.043025999 |
| OST4 | 0.04299587 |
| USP24 | 0.042364294 |
| DDX5 | 0.042316861 |
| ZNF550 | 0.042274002 |
| TRIM33 | 0.042121258 |
| RBM38 | 0.042027924 |
| C17orf61 | 0.041957933 |
| BTG1 | 0.041606496 |
| C13orf18 | 0.041557006 |
| AP2S1 | 0.041375509 |
| NBPF10 /// NBPF15 /// NBPF16 | 0.041096907 |
| GVIN1 | 0.040936661 |
| MTMR1 | 0.040930045 |
| LOC282997 | 0.040890414 |
| TMEM41B | 0.040864602 |
| SYNJ1 | 0.040739381 |
| HIST1H2AJ | 0.040657191 |
| MTERFD2 | 0.040644035 |
| TMCO6 | 0.04008654 |
| C1orf77 | 0.0400603 |
| PPCS | 0.040027178 |
| LOC100131015 | 0.040026041 |
| ANKRD10 | 0.040003669 |
| ARF1 | 0.039974721 |
| ZSCAN16 | 0.039953533 |
| HIPK2 | 0.039934209 |
| ZNF418 | 0.039748692 |
| PPOX | 0.039663959 |
| ZFAND3 | 0.039652528 |
| CBR4 | 0.039629668 |
| SOCS3 | 0.039603064 |
| SLU7 | 0.039589063 |
| CARM1 | 0.039442211 |
| LOC100131067 | 0.039358616 |
| NAAA | 0.039264474 |
| DUSP28 | 0.039078729 |
| MPEG1 | 0.039078151 |
| NCAM1 | 0.038866969 |
| CSRP1 | 0.038819756 |
| KIAA0226 | 0.038771742 |
| C19orf59 | 0.038606445 |
| RFX5 | 0.038434565 |
| FUS | 0.038419246 |
| PILRB | 0.03827172 |
| KLRC1 /// KLRC2 | 0.03817036 |
| TMEM218 | 0.03816496 |
| CYB5R1 | 0.038123263 |
| KIAA0495 | 0.038023669 |
| PITPNB | 0.03791322 |
| UBE2L3 | 0.037878989 |
| DPY19L1 | 0.03757938 |
| SFRS4 | 0.037555317 |
| FAM46A | 0.03746896 |
| CISH | 0.037263289 |
| PSMG4 | 0.037200318 |
| SFI1 | 0.037197725 |
| ZNF606 | 0.037023942 |
| FLJ39051 | 0.036957008 |
| KLRF1 | 0.036857623 |
| DIAPH1 | 0.036804135 |
| SRRM2 | 0.036682833 |
| HDGF | 0.036632622 |
| QSOX1 | 0.036585123 |
| PDLIM5 | 0.036461461 |
| POP5 | 0.036452075 |
| SLC20A1 | 0.036415232 |
| NCOA3 | 0.036382838 |
| FAM89B | 0.036344809 |
| HLA-DMB | 0.036277583 |
| KPNA2 | 0.036003341 |
| STX16 | 0.036002232 |
| C1orf174 | 0.035996439 |
| EDEM2 | 0.035858381 |
| BLNK | 0.035855041 |
| TXNDC17 | 0.035681954 |
| C1orf63 | 0.035670215 |
| PIAS3 | 0.03558584 |
| HP1BP3 | 0.035558653 |
| SFRS11 | 0.035440633 |
| HNRNPH1 | 0.035359173 |
| H2AFJ | 0.035357629 |
| BAT2L2 | 0.035324439 |
| ATP9A | 0.035249398 |
| PCSK5 | 0.035248663 |
| KDM5A | 0.035051234 |
| NUMBL | 0.034903335 |
| DHFR | 0.034857841 |
| C17orf42 | 0.034747864 |
| SLC5A3 | 0.034730425 |
| HLA-DRB1 | 0.03468595 |
| DNMBP | 0.034676932 |
| RPL6 | 0.034612254 |
| RALGAPB | 0.034562202 |
| HP /// HPR | 0.03456161 |
| SNX19 | 0.034515623 |
| LOC283588 | 0.03447213 |
| TRAPPC6B | 0.034417891 |
| TADA2B | 0.034379291 |
| STEAP4 | 0.034374666 |
| TSPO | 0.034270643 |
| DKFZp667E0512 | 0.034256719 |
| ZNF644 | 0.03403482 |
| NDC80 | 0.034030688 |
| RPL23 /// SNORA21 | 0.034000249 |
| LSMD1 | 0.033957157 |
| SH2B3 | 0.033917563 |
| CYTSA | 0.033877127 |
| CDK11A /// CDK11B | 0.033823256 |
| SEC24C | 0.033762052 |
| SPSB3 | 0.033756866 |
| FGR | 0.033721518 |
| GAPDH | 0.03362224 |
| FAM13B | 0.033184512 |
| PIM1 | 0.033142513 |
| JARID2 | 0.033052438 |
| STRBP | 0.033049598 |
| GMEB1 | 0.033031249 |
| TMED8 | 0.033027031 |
| VAT1 | 0.032967954 |
| FAM100B | 0.032896604 |
| MIR21 /// TMEM49 | 0.03274517 |
| ARHGEF7 | 0.032727478 |
| TRIM13 | 0.032703206 |
| AGAP4 /// AGAP6 /// AGAP7 /// | 0.032622119 |
| AGAP8 | |
| RUNX1 | 0.032611114 |
| NRBP1 | 0.032536658 |
| HLA-DPB1 | 0.032455803 |
| COX8A | 0.032294059 |
| SLC2A1 | 0.032293289 |
| ADCK4 | 0.032107685 |
| RAB5A | 0.032005068 |
| MED26 | 0.031988348 |
| RBMX | 0.031980495 |
| UBL4A | 0.031959408 |
| CTDSPL2 | 0.031893447 |
| POLR3E | 0.031783064 |
| ZNF14 | 0.031771392 |
| SEMA4A | 0.03175665 |
| TCF4 | 0.031712361 |
| SETDB1 | 0.03159135 |
| CTNNA1 | 0.031556267 |
| CDC34 | 0.031551822 |
| CFLAR | 0.031542314 |
| STOM | 0.031532854 |
| LOC100291860 | 0.031511157 |
| LUC7L | 0.03149945 |
| EFR3A | 0.0314806 |
| C2orf49 | 0.031446335 |
| KCTD20 | 0.031385783 |
| SFRS5 | 0.031362332 |
| QTRTD1 | 0.031344354 |
| NOG | 0.031332421 |
| CTSA | 0.031228125 |
| INPP5D | 0.031195617 |
| ODC1 | 0.031084927 |
| FRG1B | 0.030949268 |
| LFNG | 0.030917378 |
| CNOT3 | 0.030832564 |
| MARK3 | 0.030814127 |
| HCRP1 | 0.030809186 |
| SETD6 | 0.030716432 |
| SLTM | 0.030697871 |
| TAF11 | 0.030695475 |
| RBM39 | 0.030642277 |
| GPX1 | 0.03054416 |
| MBTPS1 | 0.030530655 |
| CHST15 | 0.030516331 |
| ADAM28 | 0.030449454 |
| UBE2E1 | 0.030413804 |
| STK11 | 0.030373796 |
| LASS5 | 0.030347502 |
| SLC35E1 | 0.030307811 |
| LOC100287008 | 0.030300663 |
| TOB2 | 0.030164664 |
| HSPBAP1 | 0.030160598 |
| ID2 /// ID2B | 0.03016007 |
| SNX30 | 0.029985401 |
| FBXO41 | 0.029923005 |
| PA2G4 | 0.029912905 |
| NOL9 | 0.029862716 |
| MARCHF2 | 0.029837845 |
| TMCC2 | 0.022787008 |
| ANKH | 0.029828739 |
| GBP4 | 0.029795254 |
| ZFYVE27 | 0.029759134 |
| FRY | 0.029571271 |
| GCOM1 | 0.029566864 |
| PPP2R1A | 0.029542749 |
| GALNS | 0.029484177 |
| PBX2 | 0.029475226 |
| ZDHHC6 | 0.029466145 |
| TINF2 | 0.029429249 |
| PLEK2 | 0.029423468 |
| GATAD2B | 0.029422456 |
| FAM165B | 0.029340922 |
| CREBBP | 0.029339863 |
| DPP8 | 0.029315111 |
| STK38 | 0.029312142 |
| MEPCE | 0.029288097 |
| ZYX | 0.029287272 |
| ZNF252 | 0.02927753 |
| DHRS13 | 0.029271967 |
| HBP1 | 0.029245981 |
| MIRLET7D | 0.029187297 |
| C11orf30 | 0.029102516 |
| STAMBP | 0.029054719 |
| IVNS1ABP | 0.029025623 |
| GOLGA1 | 0.029018587 |
| DAP | 0.029001413 |
| DPP3 | 0.028971031 |
| DHX9 | 0.028932947 |
| KLF11 | 0.028707257 |
| TAF1D | 0.028656026 |
| LYSMD2 | 0.028576984 |
| BLCAP | 0.028534541 |
| MTHFD2 | 0.028519398 |
| VBP1 | 0.028513711 |
| LOC643008 | 0.028503039 |
| C5orf4 | 0.028490777 |
| TRIM3 | 0.028441588 |
| ACRBP | 0.028433591 |
| ADSS | 0.028412383 |
| PTCD1 | 0.0283601 |
| PAK1 | 0.028308625 |
| TMIGD2 | 0.028260351 |
| MAP2K2 | 0.028228366 |
| NCRNA00203 | 0.028225432 |
| LMAN2L | 0.028204487 |
| ASCC2 | 0.028174308 |
| FAM111A | 0.028172592 |
| ST3GAL6 | 0.028151729 |
| COX7A2L | 0.028148094 |
| CSRP2BP /// PET117 | 0.028046167 |
| ACACB | 0.028044322 |
| LOC100289122 /// POLR2C | 0.028031291 |
| TRA2A | 0.027999763 |
| RXRA | 0.027950272 |
| TNFAIP2 | 0.027935861 |
| FAM133B | 0.027908868 |
| C10orf84 | 0.027899555 |
| FAM120AOS | 0.027802393 |
| PANK2 | 0.027758769 |
| HLA-DQA1 /// HLA-DQA2 | 0.027685758 |
| C12orf4 | 0.027651686 |
| FAM128A | 0.027649959 |
| SPCS3 | 0.027585703 |
| SAR1A | 0.027559436 |
| TMEM223 | 0.027546024 |
| CEP63 | 0.027495267 |
| EXT1 | 0.027459071 |
| NET1 | 0.027453392 |
| CD160 | 0.027446501 |
| C3orf34 | 0.02739126 |
| METTL4 | 0.027323376 |
| ATPIF1 | 0.027299239 |
| ZNF44 | 0.027210253 |
| PIM3 | 0.027141876 |
| GIMAP8 | 0.02705483 |
| TUBD1 | 0.027041874 |
| SLC31A2 | 0.027040588 |
| IRF8 | 0.027007492 |
| KIAA0141 | 0.026985681 |
| DEK | 0.026948001 |
| SMC3 | 0.026921159 |
| CLK1 | 0.026899814 |
| GTF2A1 | 0.026893963 |
| C17orf85 | 0.026793662 |
| WDSUB1 | 0.026761898 |
| PIK3R2 | 0.026729138 |
| FAM18B | 0.026720886 |
| PTGS2 | 0.026714985 |
| PPM1K | 0.026646203 |
| C5orf62 | 0.02664516 |
| GABPA | 0.026621003 |
| NDUFB11 | 0.026579319 |
| FURIN | 0.026565174 |
| CTSS | 0.026560927 |
| RBBP8 | 0.026557327 |
| LOC401320 | 0.026508925 |
| KIAA0776 | 0.026497896 |
| ZDHHC17 | 0.026410492 |
| ZNF783 | 0.026395691 |
| AMY2B /// RNPC3 | 0.026365486 |
| C4orf34 | 0.026268323 |
| R3HDM2 | 0.026266377 |
| NSUN6 | 0.026251267 |
| SENP5 | 0.026241543 |
| MCL1 | 0.026155266 |
| EIF5 | 0.026138927 |
| WAC | 0.026136142 |
| LRMP | 0.026091201 |
| SH3BGRL3 | 0.026089356 |
| MRPS18A | 0.026089087 |
| RMND5A | 0.02606981 |
| GPSM3 | 0.026069764 |
| OVOS /// OVOS2 | 0.025953058 |
| C2orf24 | 0.025926553 |
| GAS2L1 | 0.025924216 |
| DGAT2 | 0.025871875 |
| RABAC1 | 0.025870468 |
| OLFM4 | 0.025841148 |
| C3orf10 | 0.025825465 |
| NFATC2IP | 0.025772035 |
| FDFT1 | 0.025769883 |
| SPPL3 | 0.025749932 |
| MFHAS1 | 0.0257386 |
| KIAA0247 | 0.025738433 |
| ERBB2IP | 0.025720039 |
| ST3GAL5 | 0.025674603 |
| EPB41L2 | 0.025620215 |
| PRDX2 | 0.025573051 |
| DCTN2 | 0.025533761 |
| MAF1 | 0.025496326 |
| FAM156A /// FAM156B | 0.025455004 |
| ING1 | 0.025448255 |
| AFF4 | 0.025429162 |
| C14orf138 | 0.025408213 |
| OSTM1 | 0.025399212 |
| PMAIP1 | 0.025371942 |
| SLC38A2 | 0.025371649 |
| SP3 | 0.025346697 |
| DUSP18 | 0.025332365 |
| KIAA1530 | 0.025307356 |
| OSBPL8 | 0.02529967 |
| C10orf128 | 0.025292645 |
| MED6 | 0.025266554 |
| TET3 | 0.025246831 |
| LOC200030 /// NBPF1 /// NBPF10 /// | 0.02523834 |
| NBPF11 /// NBPF14 /// NBPF15 /// | |
| NBPF16 /// NBPF8 /// NBPF9 | |
| UBE3C | 0.025119256 |
| PDP1 | 0.025118073 |
| MRPL11 | 0.025073945 |
| AFF3 | 0.025072123 |
| HP | 0.025034568 |
| BAG5 | 0.025026571 |
| ERCC5 | 0.025003728 |
| FBXO11 | 0.024972942 |
| AEN | 0.024919633 |
| HMBOX1 | 0.024889272 |
| FAM8A1 | 0.024879404 |
| ARG1 | 0.024858501 |
| MAL | 0.024781655 |
| NCRNA00201 | 0.024780875 |
| FOXP1 | 0.024768806 |
| CLEC2B | 0.024760863 |
| RIT1 | 0.024744639 |
| STXBP3 | 0.024712627 |
| PPTC7 | 0.024685917 |
| ARG2 | 0.024680256 |
| TOR3A | 0.024676356 |
| FAM159A | 0.024674749 |
| HELQ | 0.024671965 |
| TMEM216 | 0.024668679 |
| PARP8 | 0.024644319 |
| ELK4 | 0.024608774 |
| SF1 | 0.024559325 |
| SETD4 | 0.024555222 |
| PATL2 | 0.024495169 |
| KDM2B | 0.024491073 |
| OSBP2 | 0.024484194 |
| TNS1 | 0.024464807 |
| STAG1 | 0.02443575 |
| PABPN1 | 0.024397211 |
| KIAA0355 | 0.024371772 |
| MRI1 | 0.024369446 |
| LAMP1 | 0.024293499 |
| AUTS2 | 0.024285452 |
| AFTPH | 0.024284622 |
| HMGN3 | 0.024279809 |
| EYA3 | 0.024279565 |
| UFD1L | 0.024234103 |
| CST7 | 0.024227579 |
| GON4L | 0.024195859 |
| HERC2 | 0.024193945 |
| LSM12 | 0.024177794 |
| LOC643837 | 0.0241777 |
| ASXL1 | 0.024149716 |
| GPRASP1 | 0.024074607 |
| IL4R | 0.024061097 |
| KRR1 | 0.024060928 |
| HIBCH | 0.024043137 |
| NSL1 | 0.024040941 |
| TMEM48 | 0.024013224 |
| SNHG12 | 0.024007956 |
| TCP11L2 | 0.023923679 |
| EMP3 | 0.023918651 |
| PL-5283 | 0.023868909 |
| TTLL12 | 0.023800015 |
| ERCC3 | 0.02379366 |
| CNDP2 | 0.023765722 |
| RNF187 | 0.023754272 |
| RIN2 | 0.023735801 |
| ANKHD1 | 0.023715077 |
| LOC375190 | 0.023689205 |
| LYL1 | 0.023651024 |
| LOC100286909 | 0.02363007 |
| THAP11 | 0.023600346 |
| C5orf32 | 0.023579584 |
| ZNF506 | 0.023567404 |
| PDCD4 | 0.023547086 |
| KSR1 | 0.023536382 |
| VIPAR | 0.023528988 |
| LMLN | 0.023507755 |
| TATDN3 | 0.023500687 |
| LAPTM4A | 0.023442636 |
| UBE2Q1 | 0.023434357 |
| SAPS3 | 0.023421516 |
| PRR5 | 0.023421199 |
| PLEKHA2 | 0.023383955 |
| SDHAF2 | 0.023370467 |
| LOC100287331 | 0.023358376 |
| C6orf1 | 0.02332283 |
| VWCE | 0.023294508 |
| ROMO1 | 0.023242583 |
| CELF2 | 0.023213121 |
| RGS2 | 0.023212391 |
| ASPH | 0.023135961 |
| KIAA1310 | 0.023135645 |
| PRMT2 | 0.023129954 |
| GPATCH8 | 0.023128358 |
| NBR1 | 0.023087349 |
| UFM1 | 0.023082457 |
| PICALM | 0.02307674 |
| SNHG1 | 0.023033207 |
| GLIPR1 | 0.022997058 |
| ARPC3 | 0.02296092 |
| FAM122B | 0.022951056 |
| YIPF2 | 0.022889275 |
| DNAJB2 | 0.022864826 |
| VPS24 | 0.022860337 |
| LUZP6 /// MTPN | 0.022856481 |
| RFT1 | 0.022845402 |
| TMPO | 0.022820514 |

TABLE 7
Top 500 Transcripts ranked according to the Fisher's score
Patients Positive and Negative for anti-dsDNA Antibodies

| Gene | Fisher Score |
|---|---|
| CLEC4D | 0.248228776 |
| LGALS3BP | 0.240224585 |
| USP18 | 0.238664565 |
| SPATS2L | 0.235055761 |
| EEF1G /// TUT1 | 0.226539848 |
| NT5C3 | 0.224247056 |
| KLHDC7B | 0.21749338 |
| MT1G | 0.210089445 |
| GTPBP2 | 0.195499524 |
| RPH3A | 0.195081509 |
| ZBP1 | 0.189431277 |
| RPL13A /// RPL13AP5 | 0.189129482 |
| RTP4 | 0.188867949 |
| EIF2AK2 | 0.187942356 |
| IGLV3-19 | 0.187548665 |
| SP140 | 0.185031803 |
| TNFSF10 | 0.183914789 |
| MT1H /// MT1P2 | 0.180142494 |
| IFI27 | 0.178579236 |
| HIST1H2BD | 0.178372047 |
| HERC6 | 0.174773352 |
| TDRD7 | 0.173506982 |
| MAP2K6 | 0.17327457 |
| IL1RN | 0.173004219 |
| PARP9 | 0.172324763 |
| TRIM21 | 0.172300281 |
| PDZD4 | 0.171458765 |
| CEACAM1 | 0.171267281 |
| TOR1B | 0.171223338 |
| CAMK2N1 | 0.170941348 |
| OASL | 0.170247639 |
| CIITA | 0.169348156 |
| CCRL2 | 0.167719012 |
| HLA-DMB | 0.166843733 |
| GALM | 0.165137401 |
| IFI6 | 0.164445743 |
| MT1P2 | 0.163191522 |
| TIMM10 | 0.162018066 |
| DHX58 | 0.161942311 |
| EEF2 | 0.161086418 |
| IFIH1 | 0.158801701 |
| IRF7 | 0.158586108 |
| AHNAK | 0.156762914 |
| ISG15 | 0.155286255 |
| RPL3 | 0.153936105 |
| LY6E | 0.153549551 |
| EEF1B2 | 0.151475436 |
| CD1C | 0.151429246 |
| GLG1 | 0.1478886 |
| UBE2L6 | 0.147759729 |
| AMPD2 | 0.147165792 |
| NTNG2 | 0.145362528 |
| CABC1 | 0.144826704 |
| TRIM5 | 0.144777137 |
| DHRS9 | 0.14419429 |
| IL18R1 | 0.143724939 |
| BCL2A1 | 0.142120508 |
| OR52K3P | 0.142081834 |
| LOC339988 | 0.141830865 |
| RPL7 | 0.141544843 |
| ZCCHC2 | 0.140682923 |
| RPL5 | 0.139754466 |
| SHISA5 | 0.138893919 |
| LOC100294182 /// RPL3 | 0.137987875 |
| TRIM69 | 0.137882602 |
| FLJ42418 | 0.13715254 |
| TRAFD1 | 0.137110829 |
| AKIRIN2 | 0.136228356 |
| PARP12 | 0.136121947 |
| AIM2 | 0.135461855 |
| NCRNA00183 | 0.135232019 |
| MOBKL2C | 0.13490582 |
| PABPC1 | 0.133989159 |
| SIGLEC1 | 0.133636898 |
| HIST1H2BE | 0.13357855 |
| GPR183 | 0.132905943 |
| TMEM140 | 0.132281646 |
| GADD45B | 0.13136474 |
| LOC731424 | 0.131197038 |
| ZC3HAV1 | 0.130795304 |
| IFIT5 | 0.130571883 |
| MID1 | 0.129912271 |
| IFI35 | 0.129907032 |
| ADAR | 0.129873842 |
| H2BFS | 0.129627837 |
| MX1 | 0.129401745 |
| EIF4B | 0.12902905 |
| CACNA1A | 0.128218021 |
| SERPING1 | 0.128002938 |
| PABPC4 | 0.127643172 |
| PLSCR1 | 0.12735805 |
| TCN1 | 0.127022409 |
| SAMD9 | 0.126868225 |
| SRBD1 | 0.126368776 |
| ZNF264 | 0.125979886 |
| MOV10 | 0.125837913 |
| C18orf49 | 0.124940112 |
| TRIM66 | 0.1248108 |
| HIST1H2AJ | 0.124784953 |
| TOR1A | 0.12394714 |
| LOC727751 /// LOC727849 /// | 0.123881466 |
| LOC80154 | |
| CYSLTR1 | 0.123144344 |
| SP110 | 0.12219326 |
| CAND2 | 0.122173519 |
| IFI44 | 0.1220071 |
| MT2A | 0.121551478 |
| GABBR1 | 0.121539777 |
| GPD2 | 0.121310667 |
| RPS14 | 0.12122247 |
| GTPBP1 | 0.12081732 |
| LOC439949 | 0.12062477 |
| FAM113A | 0.12049443 |
| C1GALT1 | 0.119803534 |
| CAMK1D /// LOC283070 | 0.119459402 |
| COQ10A | 0.119015915 |
| MT1E | 0.118653776 |
| HIST1H2BC | 0.118002181 |
| ACOT13 | 0.117910216 |
| IFI16 | 0.117774695 |
| ZNF230 | 0.117626342 |
| BST2 | 0.117610811 |
| OAS2 | 0.11705334 |
| IGK@ /// IGKC /// LOC652493 | 0.116891954 |
| HIST2H2AA3 /// HIST2H2AA4 | 0.116735847 |
| MX2 | 0.116622278 |
| HIST2H2AA3 | 0.116558325 |
| FTSJD2 | 0.116515179 |
| LOC100286937 /// RASA4 | 0.11642819 |
| ANKS6 | 0.115814845 |
| LOC100287723 | 0.115612099 |
| TAP1 | 0.115402729 |
| ZNF703 | 0.115187458 |
| ALOX5AP | 0.115157799 |
| HIST2H2BE | 0.114921596 |
| TOMM20 | 0.114692434 |
| IL7R | 0.11446112 |
| SLC26A8 | 0.114304282 |
| RPL13A | 0.113919994 |
| PRPF31 | 0.113379955 |
| CPVL | 0.112791291 |
| SMTNL1 | 0.111949573 |
| APOL6 | 0.111418345 |
| DDAH2 | 0.111253655 |
| GBP1 | 0.111041697 |
| CCR1 | 0.110578774 |
| CMPK2 | 0.110521707 |
| HSH2D | 0.110083109 |
| CREBL2 | 0.10982763 |
| LAP3 | 0.109557423 |
| IGH@ /// IGHG1 /// IGHM /// | 0.109346289 |
| IGHV4-31 /// LOC100290146 | |
| IGH@ /// IGHA1 /// IGHA2 /// | 0.109310407 |
| LOC100126583 | |
| DKFZp761E198 | 0.109293775 |
| RHOT2 | 0.10922301 |
| TMEM123 | 0.108962296 |
| IFIT2 | 0.108789453 |
| IFI44L | 0.108187896 |
| OAS3 | 0.107826507 |
| LOC100290557 | 0.107746864 |
| RAB8A | 0.107727863 |
| HIST1H2AC | 0.106953811 |
| SAMD9L | 0.106930354 |
| IFIT1 | 0.106765551 |
| FFAR2 | 0.106559288 |
| LAMP3 | 0.105504955 |
| PML | 0.105251742 |
| XAF1 | 0.105049422 |
| RPS23 | 0.104966905 |
| KLHDC8B | 0.104751204 |
| ASB1 | 0.10468811 |
| SPTLC2 | 0.104647334 |
| LOC100130357 | 0.10458974 |
| KIAA1147 | 0.104539328 |
| IGLV2-23 /// LOC100293440 | 0.104527054 |
| C3AR1 | 0.104458594 |
| IRF9 | 0.104339724 |
| TRIM14 | 0.104209841 |
| ERCC1 | 0.104177145 |
| PI3 | 0.103481618 |
| RNF213 | 0.102961234 |
| EIF3L | 0.102945235 |
| CAPG | 0.102833724 |
| LOC729317 /// VDAC2 | 0.102784405 |
| HERC5 | 0.102443219 |
| PARP14 | 0.102424897 |
| HIST1H4H | 0.102327522 |
| C18orf25 | 0.102274261 |
| VPS37C | 0.101900226 |
| LOC100289090 | 0.101547449 |
| CSDE1 | 0.101354628 |
| CSF1R | 0.100797324 |
| BUD31 | 0.100678645 |
| FBXO6 | 0.100533388 |
| RBCK1 | 0.100526976 |
| DDX60 | 0.099867343 |
| HIST1H2AD /// HIST1H3D | 0.099816427 |
| SRA1 | 0.099665237 |
| KCTD7 | 0.099601737 |
| HMGB2 | 0.099486479 |
| PRRG4 | 0.099245954 |
| MFNG | 0.099188773 |
| LOC100293440 | 0.099021522 |
| EPSTI1 | 0.098462736 |
| BST1 | 0.098272056 |
| MTERFD3 | 0.098073338 |
| ZFHX3 | 0.097926678 |
| RSAD2 | 0.097684831 |
| IGLV1-44 /// LOC100290557 | 0.097590013 |
| LOC652493 | 0.097567706 |
| C11orf2 | 0.097111112 |
| RPS7 | 0.096900953 |
| ZNF395 | 0.0968715 |
| FAM46A | 0.096615259 |
| LOC284023 | 0.096548113 |
| SLFN12 | 0.096446232 |
| IGK@ /// IGKC /// LOC100291464 | 0.096435918 |
| AIF1 | 0.096410256 |
| ACACB | 0.096263668 |
| SNRNP200 | 0.096004429 |
| DYNLT1 | 0.095848312 |
| MRPS27 | 0.095798273 |
| NPM1 | 0.095278164 |
| RBM8A | 0.09477615 |
| TMEM80 | 0.094770535 |
| EIF3D | 0.094566498 |
| G0S2 | 0.094468181 |
| BATF2 | 0.094453972 |
| TIAM1 | 0.094450506 |
| CARD6 | 0.094194212 |
| CARD16 /// CASP1 | 0.094162178 |
| NMI | 0.09407601 |
| EEF1A1 /// EEF1A1P9 | 0.094042293 |
| IGK@ /// IGKC /// LOC652493 /// | 0.093926197 |
| LOC652694 | |
| TRIT1 | 0.093875343 |
| RAB11FIP3 | 0.093848399 |
| ANKHD1 | 0.093772957 |
| PABPC3 | 0.093721678 |
| LOC200772 | 0.093597917 |
| GOLGA8A | 0.093575793 |
| CXorf21 | 0.09355694 |
| SET | 0.093453178 |
| DTX3L | 0.09315357 |
| SNRNP70 | 0.093062254 |
| SFRS11 | 0.093027804 |
| GPR68 | 0.092746882 |
| PRPF4B | 0.092742916 |
| KLRB1 | 0.092461081 |
| PKP4 | 0.092269685 |
| CD6 | 0.092248284 |
| FAM8A1 | 0.092214441 |
| OAS1 | 0.092179441 |
| CKAP4 | 0.092086275 |
| HIATL1 | 0.09206292 |
| FBL | 0.09194182 |
| LDHB | 0.091827969 |
| NUP133 | 0.091793523 |
| APOL2 | 0.091743793 |
| MYB | 0.091715106 |
| FLJ36031 | 0.091476882 |
| PDCD2 | 0.091459888 |
| RPLP0 | 0.091434686 |
| PTGDS | 0.091368309 |
| LTA4H | 0.091310607 |
| PID1 | 0.091269705 |
| TXNL4B | 0.091168817 |
| IGLC1 /// IGLL5 /// IGLV3-16 /// | 0.090765624 |
| IGLV3-25 | |
| HNRNPA1 | 0.090715163 |
| IGK@ /// IGKC /// IGKV3D-15 | 0.090685422 |
| ZBTB4 | 0.090555852 |
| C5orf62 | 0.090443157 |
| RPL9 | 0.090167515 |
| SIGLEC10 /// SIGLEC12 | 0.090133917 |
| MOAP1 | 0.090124208 |
| ENO2 | 0.089810375 |
| CAMP | 0.08971231 |
| DPP4 | 0.089529326 |
| PSTPIP2 | 0.089515835 |
| IGL@ | 0.089489876 |
| OXA1L | 0.08943573 |
| STUB1 | 0.089385963 |
| SRRM2 | 0.089345415 |
| RPL6 | 0.08922766 |
| CASP10 | 0.089174062 |
| PAN2 | 0.089143203 |
| TRIM22 | 0.088855424 |
| TPT1 | 0.088836166 |
| PI4KA | 0.088685879 |
| TREX1 | 0.08867143 |
| RPL23A | 0.088116565 |
| AGAP4 /// AGAP6 /// AGAP7 /// | 0.087779114 |
| AGAP8 | |
| IGHA1 /// IGHD /// IGHG1 /// | 0.08773785 |
| IGHG3 /// IGHM /// IGHV4-31 /// | |
| LOC100290320 /// LOC100291190 | |
| LOC100130100 /// LOC100291464 | 0.087600729 |
| STMN3 | 0.087564275 |
| TNFRSF17 | 0.087524542 |
| CRISP3 | 0.087095027 |
| PSME1 | 0.087067066 |
| MYD88 | 0.087050408 |
| TBC1D9 | 0.086976076 |
| PCYOX1L | 0.086809599 |
| IFIT3 | 0.086740128 |
| SPOCK2 | 0.086509604 |
| LOC100132999 | 0.086151269 |
| PNPT1 | 0.085837694 |
| STAT2 | 0.085711598 |
| TNFAIP6 | 0.085368316 |
| CD4 | 0.08480652 |
| ZNF248 | 0.084720745 |
| RPL34 | 0.084711265 |
| PAQR8 | 0.084557176 |
| SIPA1L2 | 0.084492298 |
| PTEN | 0.084429921 |
| SRGAP2P1 | 0.084425339 |
| HLA-DRB1 | 0.084320763 |
| MT1X | 0.084216108 |
| MSRB2 | 0.083548424 |
| IL11RA | 0.082999113 |
| WHSC2 | 0.082777128 |
| ENGASE | 0.082639819 |
| MGC16275 | 0.082546282 |
| DDX60L | 0.082506215 |
| TRIM56 | 0.082459584 |
| MGC29506 | 0.082246465 |
| MRI1 | 0.082194815 |
| PEA15 | 0.081912292 |
| MAP3K12 | 0.08188525 |
| CPSF7 | 0.081851179 |
| SERTAD2 | 0.081436057 |
| RPL10A | 0.081359576 |
| TMEM62 | 0.080908705 |
| TNFSF13B | 0.080868941 |
| PTP4A1 | 0.080554682 |
| SRGAP2 | 0.080531483 |
| LILRA5 | 0.080360136 |
| TLR2 | 0.080231196 |
| API5 | 0.080142026 |
| FAM153A /// FAM153B /// FAM153C | 0.080018186 |
| LINS1 | 0.08001327 |
| SNRPN | 0.07989276 |
| HMGN3 | 0.079691514 |
| ASXL1 | 0.079633334 |
| PSMB4 | 0.079517659 |
| C10orf12 | 0.079403851 |
| FAM125A | 0.079309233 |
| CYAT1 /// IGLV1-44 | 0.079119007 |
| SUN1 | 0.078747335 |
| HLA-DRB1 /// HLA-DRB4 | 0.078738923 |
| DIMT1L | 0.078638704 |
| CHIC2 | 0.078578388 |
| MGC27345 | 0.078538801 |
| IGL V2-23 | 0.078319854 |
| FOXO1 | 0.078319607 |
| FTO | 0.078138523 |
| S100A6 | 0.078002378 |
| ALG13 | 0.077877776 |
| CD44 | 0.077745175 |
| MOSC1 | 0.077197653 |
| DAPP1 | 0.077109575 |
| TLK2 | 0.077031143 |
| DISC1 /// TSNAX-DISC1 | 0.076903045 |
| PI4KA /// PI4KAP1 /// PI4KAP2 | 0.076884796 |
| DLG5 | 0.076667689 |
| NFIL3 | 0.076604262 |
| IGLV1-44 | 0.076414343 |
| AZI2 | 0.076279042 |
| NFATC3 | 0.076238906 |
| TRIM38 | 0.076144022 |
| TYMP | 0.076131444 |
| EIF3F | 0.075974688 |
| ACOT9 | 0.075969183 |
| SMARCA4 | 0.075914656 |
| FBLN7 | 0.075835149 |
| MUTED /// TXNDC5 | 0.075688333 |
| PPP2R5C | 0.075675695 |
| FAM168B | 0.075645038 |
| NDUFV1 | 0.075637432 |
| ZNF573 | 0.075590827 |
| KLF13 | 0.075568122 |
| RPS13 | 0.07555626 |
| RPL27 | 0.075438559 |
| PEX5 | 0.075314018 |
| IFITM3 | 0.074902313 |
| EIF3K | 0.074857739 |
| C19orf66 | 0.074793433 |
| TRANK1 | 0.074773436 |
| IGLJ3 | 0.074591319 |
| AGRN | 0.074474885 |
| IFITM1 | 0.074430274 |
| RPS3A | 0.074364108 |
| RNF214 | 0.074356887 |
| IGKV4-1 | 0.074353169 |
| JMJD7-PLA2G4B /// PLA2G4B | 0.074310913 |
| TMEM50B | 0.074264425 |
| ADCY4 | 0.074141513 |
| STAT1 | 0.07396559 |
| RPL26 | 0.073947226 |
| CNP | 0.073879696 |
| RNMT | 0.073855601 |
| IARS2 | 0.07372147 |
| IGJ | 0.073593559 |
| CARD16 | 0.073418039 |
| TSC1 | 0.073256654 |
| ZNF420 | 0.073162206 |
| CLEC4E | 0.07315168 |
| RNF220 | 0.073075641 |
| LGALS9 | 0.073030772 |
| METT10D | 0.072883801 |
| GOLGA6L4 /// PML | 0.072881002 |
| HTATIP2 | 0.072833819 |
| CASP1 | 0.072595669 |
| TLE4 | 0.072468978 |
| AKR7A2 | 0.072399152 |
| FAM91A2 /// FLJ39739 /// | 0.072284406 |
| LOC100132057 /// LOC100286793 /// | |
| LOC728855 /// LOC728875 | |
| IGK@ /// IGKC /// IGKV3-20 /// | 0.072052107 |
| LOC100291682 | |
| C16orf80 | 0.071919871 |
| IGH@ /// IGHA1 /// IGHA2 /// | 0.071910522 |
| IGHG1 /// IGHG2 /// IGHG3 /// | |
| IGHM /// IGHV4-31 /// | |
| LOC100126583 /// LOC100290036 | |
| HVCN1 | 0.07175294 |
| CMPK1 | 0.071611278 |
| SBNO1 | 0.071159814 |
| KIAA0226 | 0.071159062 |
| YLPM1 | 0.071043397 |
| OSGEPL1 | 0.070939488 |
| UPF3A | 0.070883375 |
| PPM1L | 0.070819852 |
| NCOA7 | 0.070760046 |
| DNAJC27 | 0.070522684 |
| AUTS2 | 0.070327173 |
| XRN1 | 0.070278532 |
| MED6 | 0.070160069 |
| ZNF606 | 0.070114216 |
| SBDS /// SBDSP1 | 0.069966692 |
| DNAJA3 | 0.069583723 |
| LCN2 | 0.069483485 |
| MT1F | 0.069458118 |
| POLR2B | 0.069443851 |
| SETD7 | 0.069406704 |
| PSMB9 | 0.069370165 |
| TBCD | 0.069358117 |
| NCRNA00201 | 0.069327442 |
| MRPL20 | 0.069272745 |
| LETMD1 | 0.069215821 |
| PDK4 | 0.068863981 |
| LOC100287887 /// RPL13A /// | 0.068716874 |
| RPL13AP20 /// RPL13AP3 /// | |
| RPL13AP5 /// RPL13AP6 | |
| SLC25A26 | 0.068676868 |
| ST13 | 0.068640076 |
| IDH3B | 0.06861124 |
| HLA-DMA | 0.068544384 |
| TPK1 | 0.068506935 |
| SNHG8 | 0.068502955 |
| CHN2 | 0.068488605 |
| RPL19 | 0.068389533 |
| DPH3 | 0.068305637 |
| KIF1B | 0.068264704 |
| FAM65A | 0.068179648 |
| MCCC1 | 0.068175972 |
| UQCRB | 0.068165076 |
| RPL7A | 0.068097646 |
| PHF11 | 0.067980744 |
| CDK14 | 0.067862256 |
| HAUS5 | 0.067853101 |
| ARHGEF18 | 0.067767449 |
| RPL4 | 0.067699685 |
| SUMF1 | 0.067511939 |
| KIAA0114 | 0.067508238 |
| SNHG12 | 0.067481393 |
| TNFAIP3 | 0.067431502 |
| LOC283588 | 0.067310019 |
| TMED10 | 0.067271283 |
| BRI3BP | 0.067223196 |
| B3GNT2 | 0.067210603 |
| VMA21 | 0.067149552 |
| TTC15 | 0.066895878 |
| RPS8 | 0.066869344 |
| SETD6 | 0.066805048 |
| ZNF438 | 0.066712617 |
| RPS14P3 | 0.066712043 |
| BAK1 | 0.066652589 |
| LOC93622 | 0.066639977 |
| PI4K2B | 0.066579284 |
| MDFIC | 0.066427571 |
| MAP1D | 0.066422406 |
| CPEB2 | 0.066412622 |
| HPS4 | 0.06637011 |
| LOC541471 /// NCRNA00152 | 0.066335781 |
| SGPL1 | 0.066327024 |
| HIST1H2BK | 0.066291923 |
| NDST2 | 0.066264101 |
| PELP1 | 0.066257532 |
| AKT2 | 0.066109397 |
| LRIG2 | 0.066107469 |
| ETV7 | 0.066036736 |
| WDR6 | 0.065992553 |
| RPS27A | 0.065875367 |
| ZBTB40 | 0.06574484 |
| ZNF589 | 0.065729524 |
| CHMP5 | 0.065626936 |
| RBM19 | 0.065543499 |
| IGKC | 0.065494296 |
| DIP2B | 0.065479726 |
| SLC7A6 | 0.065366635 |
| IGLL1 /// IGLL3 /// LOC91316 | 0.065252206 |
| WWP1 | 0.065231472 |

To further determine the biological significance of anti-TFAM antibody, anti-dsDNA antibody, and thrombosis PC1, enrichment analysis was performed on the transcripts that were significantly correlated with each component. It was found that 1799 and 246 transcripts were positively and negatively correlated with anti-TFAM PC1. Supplemental File s1) Positively enriched transcripts were linked to pathways associated with immune-mediated activation and hemostasis, such as response to virus and cytokine stimuli, neutrophil degranulation, activation of the innate immune response, vascular endothelial growth factor A (VEGFA)-VEGFR2 signaling, responses to bacteria, cellular response to stress, hemostasis, and autophagy, among others. In contrast, transcripts that were negatively correlated were mainly associated with pathways linked to RNA and DNA metabolism, including protein translation, RNA localization and splicing, chromatin organization, RNP complex biogenesis, cell cycle, and mitochondrial gene expression, among others (FIG. 5E).

As indicated by the strong correlation between anti-TFAM antibodies and thrombosis PC1s (FIG. 5C), the enrichment of both components was essentially identical, with the exception of two pathways related to response to virus and cytokine stimuli (FIG. 5E). Notably, pathways involving hemostasis and VEGFA-VEGFR2 signaling, as well as RNA and DNA metabolism (except for RNA translation and protein modification) were exclusively linked to anti-TFAM antibodies and thrombosis, whereas type I IFN was only associated with anti-dsDNA antibodies (FIG. 5E). Other immune-mediated activation pathways showed a significant similarity between anti-TFAM antibodies and thrombosis with anti-dsDNA antibodies (FIG. 5E), which is most likely explained by the 30% overlap between anti-dsDNA and anti-TFAM antibodies. Collectively, the data support that anti-TFAM antibodies are mechanistically related to thrombosis in SLE, but are independent to hallmark features, such as disease activity, anti-dsDNA antibodies and the IFN signature. Consistent with these findings, circulating activity levels of IFN-I or IFN-II were not associated with anti-TFAM antibodies (FIG. 5F and FIG. 5G, respectively). Unexpectedly, however, IFN-III was significantly elevated in patients positive for antibodies to TFAM (p=0.0001) (FIG. 5H).

### DISCUSSION

Mitochondria appear to play at least two immunogenic and potentially independent roles in SLE pathogenesis. First, nucleoids—particularly containing Ox mtDNA—have been linked to the induction of IFN-I by pDCs and mtDNA is considered an immunogenic candidate for the production of anti-dsDNA antibodies in SLE (Bennett et al., 2003; Reimer et al., 1984). In this context, mitochondria may have a mechanistic role in clinical phenotypes associated with the IFN signature and disease activity features related to anti-dsDNA antibodies, such as complement activation and lupus nephritis. Second, antibodies to mitochondrial proteins (e.g., 60-kd heat-shock protein, HSP60) and cardiolipin-protein complexes have been associated with thrombosis (Dieude et al., 2011; Petri, 2020), supporting a role for non-mtDNA-associated components in thrombotic events. Because TFAM is the main nucleoprotein involved in the packing of mtDNA into nucleoids (Marchi et al., 2023; Fisher et al., 1985; Parisi et al., 1991), and Ox nucleoids have been linked to the induction of IFN-I by pDCs in SLE (Bennett et al., 2003), anti-TFAM antibodies and their relationship to antibodies to DNA and the IFN signature is particularly interesting. Collectively, the clinical and transcriptional data show that anti-TFAM antibodies are not mechanistically linked to antibodies to dsDNA or IFN-I induced activation, but rather constitute a novel biomarker associated with APS and thrombosis in SLE. Indeed, the findings that antibodies to TFAM are also present in patients with primary APS supports the notion that these antibodies are generated in response to mechanisms of mitochondrial damage other than those potentially associated with anti-dsDNA and IFN-I induction.

While antibodies to TFAM and dsDNA overlap in about a third of patients with SLE, they identify two distinct clinical and transcriptional subsets within SLE. Anti-dsDNA antibodies are associated with mechanisms related to IC formation, including complement activation, nephritis, toll-like receptor (TLR) signaling and IFN-I production. In contrast, anti-TFAM antibodies are linked to thrombotic events, APS and thrombosis-associated transcriptional fingerprints, but not to pathways activated by IFN-I. Indeed, unlike SLE-associated autoantibodies such as anti-dsDNA, anti-Sm, anti-RNP, anti-DNase1L3 and anti-Ro52Ex4, which are strongly associated with high levels of circulating IFN-I (Gomez-Banuelos et al., 2024; Oke et al., 2019), antibodies to TFAM showed no relationship with IFN-I levels. These data are consistent with the lack of correlation between APS-associated autoantibodies and IFN-I levels, or the IFN-I signature (Petri et al., 2019; Gomez-Banuelos et al., 2024; Oke et al., 2019). Thus, although TFAM is the major carrier of mtDNA in nucleoids, the findings herein indicate that the immune response to TFAM in SLE is independent of any potential role of mtDNA in anti-dsDNA antibody production, and anti-TFAM antibodies appear to play no role in the induction of IFN-I by cell-free nucleoids. Nevertheless, it is worth noting that antibodies to TFAM were associated with increased activity levels of IFN-III. Although it is unclear whether IFN-III promotes the production of anti-TFAM antibodies or these antibodies induce IFN-III expression, and whether IFN-III is relevant for thrombosis, an association between IFN-λ1 and thrombotic events has been suggested in SLE, but it was not statistically significant (Oke et al., 2019). The mechanistic relationship between anti-TFAM antibodies, IFN-III, and APS has yet to be determined.

Notably, the risk of thrombosis associated with anti-TFAM antibodies is similar to and independent of LAC, the stronger risk factor for thrombosis in SLE (Galli et al., 2003; Petri et al., 1987; Wahl et al., 1997; Akhter et al., 2013). Moreover, a history of smoking or LAC has an additive effect on the risk of thrombosis in SLE patients positive for anti-TFAM antibodies, implying that these antibodies are markers of thrombotic pathways distinct from those associated with smoking and LAC. Indeed, while the prothrombotic effect of LAC is mediated by targeting the coagulation pathway (Petri et al., 2020; Pengo, 2022), the presence of antibodies to TFAM indicate thrombotic mechanisms involving cellular and mitochondrial damage.

Although the study of neutrophils provided initial clues for the search of antibodies to TFAM, the cellular source of TFAM that drives the production of autoantibodies may have different origins in SLE, including neutrophils, erythrocytes and platelets. For instance, TFAM released from IFN-I activated neutrophils in response to RNP IC stimulation (Bennett et al., 2003), as well as by forms of cell death involving mitochondrial membrane permeabilization, such as pyroptosis, apoptosis and necrosis (Miao et al., 2023; Tian et al., 2022), are potential sources of immunogenic TFAM in SLE. Interestingly, although mtDNA is found in neutrophil extracellular traps (NETs) (Lood et al., 2016), TFAM has not been detected in NETs by mass spectrometry analysis (Petretto et al., 2019; Bruschi et al., 2019). The abnormal accumulation of erythrocytes carrying mitochondria in SLE and their clearance by macrophages may also trigger the production of antibodies to TFAM (Caielli et al., 2021). Lastly, extracellular mitochondria released from IC-activated platelets have been proposed as a key source of mitochondrial antigens in SLE (Melki et al., 2021).

Interestingly, mitochondria are essential for platelet function and therefore, mitochondrial damage or dysfunction can affect platelet survival and increase the risk of thrombosis (Melchinger et al., 2019). In this case, the production of anti-TFAM antibodies may represent markers of mitochondrial dysfunction associated with thrombosis rather than direct drivers of thrombotic events in SLE. Alternatively, these antibodies may promote thrombosis by binding TFAM-DNA complexes released from activated neutrophils, degranulating platelets, apoptotic cells undergoing secondary necrosis, or from other forms of cell death. In either scenario, as markers of prothrombotic mitochondrial dysfunction or direct triggers of thrombosis, the discovery of antibodies to TFAM provides a novel tool independent of traditional APS-associated antibodies for identifying SLE patients at risk of thrombosis, where mitochondrial damage is likely to play a pathogenic role.

### Results

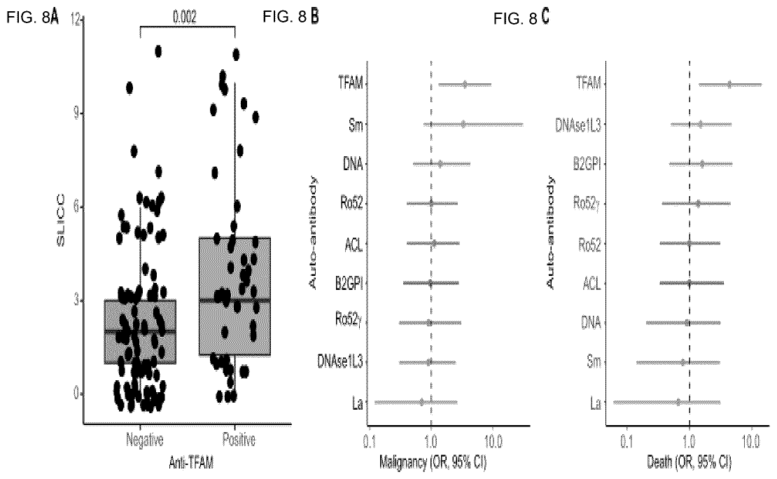

Thirty percent (48/158) of SLE patients were positive for anti-TFAM antibodies. Anti-TFAM positive patients had higher SLICC scores compared to anti-TFAM negative patients (3.9 vs 2.3) (FIG. 8A). Malignancy was the most frequent subdomain linked with elevated SLICC in anti-TFAM positive patients (29%), followed by diabetes mellitus (21%), ruptured tendon (15%), pericarditis (8%), muscular atrophy (8%), skin ulcers (8%), and deep vein thrombosis (6%). Among other SLE-related autoantibodies (FIG. 8B), only anti-TFAM antibodies were associated with a greater risk of malignancy (OR 3.5). Anti-TFAM antibodies were not linked to any specific type of cancer. Intriguingly, there was no association between malignancy and thrombotic events in anti-TFAM positive patients. Furthermore, 23% (11/47) of anti-TFAM SLE patients died during follow-up (OR 4.4, FIG. 8C). The most common cause of death in anti-TFAM positive SLE were cardiovascular related in 36% (4/11).

### CONCLUSIONS

Anti-TFAM antibodies identify a subset of patients with SLE at higher risk for unfavorable outcomes, including malignancy and death. The lack of association between malignancy and thrombotic events in anti-TFAM positive patients suggests the existence of at least two subsets of anti-TFAM antibodies of distinct significance (i.e., thrombosis-related vs. cancer-related). The higher mortality among anti-TFAM positive SLE patients, with cardiovascular causes being the leading factor, highlights the importance of developing targeted interventions to prevent adverse outcomes in patients with anti-TFAM antibodies.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

### SEQUENCE LISTING

SEQ ID NO 1
LENGTH: 246
TYPE: human TFAM Antibody

MAFLRSMWGVLSALGRSGAELCTGCGSRLRSPFSFVYLPRWFSSV
LASCPKKPVSSYLRFSKEQLPIFKAQNPDAKTTELIRRIAQRWRE
LPDSKKKIYQDAYRAEWQVYKEEISRFKEQLTPSQIMSLEKEIMD
KHLKRKAMTKKKELTLLGKPKRPRSAYNVYVAERFQEAKGDSPQE
KLKTVKENWKNLSDSEKELYIQHAKEDETRYHNEMKSWEEQMIEV
GRKDLLRRTIKKQRKYGAEEC

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.
1. Gray M W, Burger G, Lang B F. The origin and early evolution of mitochondria. Genome Biol. 2001; 2: REVIEWS1018.
2. Marchi S, Guilbaud E, Tait S W G, Yamazaki T, Galluzzi L. Mitochondrial control of inflammation. Nat Rev Immunol. 2023; 23: 159-173.
3. Fisher R P, Clayton D A. A transcription factor required for promoter recognition by human mitochondrial RNA polymerase. Accurate initiation at the heavy- and light-strand promoters dissected and reconstituted in vitro. J Biol Chem. 1985; 260: 11330-11338.
4. Parisi M A, Clayton D A. Similarity of human mitochondrial transcription factor 1 to high mobility group proteins. Science. 1991; 252: 965-969.
5. Kukat C, Larsson N G. mtDNA makes a U-turn for the mitochondrial nucleoid. Trends Cell Biol. 2013; 23: 457-463.
6. Andreeva L, Hiller B, Kostrewa D, et al. cGAS senses long and HMGB/TFAM-bound U-turn DNA by forming protein-DNA ladders. Nature. 2017; 549: 394-398.
7. Zhong Z, Liang S, Sanchez-Lopez E, et al. New mitochondrial DNA synthesis enables NLRP3 inflammasome activation. Nature. 2018; 560: 198-203.
8. Crouser E D, Shao G, Julian M W, et al. Monocyte activation by necrotic cells is promoted by mitochondrial proteins and formyl peptide receptors. Crit Care Med. 2009; 37: 2000-2009.
9. Julian M W, Shao G, Bao S, et al. Mitochondrial transcription factor a serves as a danger signal by augmenting plasmacytoid dendritic cell responses to DNA. J. Immunol. 2012; 189: 433-443.
10. Julian M W, Shao G, Vangundy Z C, Papenfuss T L, Crouser E D. Mitochondrial transcription factor A, an endogenous danger signal, promotes TNFalpha release via RAGE- and TLR9-responsive plasmacytoid dendritic cells. PLoS One. 2013; 8: e72354.
11. Little J P, Simtchouk S, Schindler S M, et al. Mitochondrial transcription factor A (Tfam) is a pro-inflammatory extracellular signaling molecule recognized by brain microglia. Mol Cell Neurosci. 2014; 60: 88-96.
12. Ahmad A, Sundquist K, Palmer K, et al. Risk prediction of recurrent venous thromboembolism: a multiple genetic risk model. J Thromb Thrombolysis. 2019; 47: 216-226.
13. Rahman A, Isenberg D A. Systemic lupus erythematosus. N. Engl. J Med. 2008; 358: 929-939.
14. Bennett L, Palucka A K, Arce E, et al. Interferon and granulopoiesis signatures in systemic lupus erythematosus blood. J. Exp. Med. 2003; 197: 711-723.
15. Caielli S, Athale S, Domic B, et al. Oxidized mitochondrial nucleoids released by neutrophils drive type I interferon production in human lupus. J Exp. Med. 2016; 213: 697-713.
16. Petri M, Fu W, Ranger A, et al. Association between changes in gene signatures expression and disease activity among patients with systemic lupus erythematosus. BMC Med Genomics. 2019; 12: 4.
17. Kirou K A, Lee C, George S, et al. Activation of the interferon-alpha pathway identifies a subgroup of systemic lupus erythematosus patients with distinct serologic features and active disease. Arthritis Rheum. 2005; 52: 1491-1503.
18. Gomez-Banuelos E, Wahadat M J, Li J, et al. Alternative exon usage in TRIM21 determines the antigenicity of Ro52/TRIM21 in systemic lupus erythematosus. JCI Insight. 2022; 7: e163795.
19. Gomez-Banuelos E, Yu Y, Li J, et al. Affinity maturation generates pathogenic antibodies with dual reactivity to DNase1L3 and dsDNA in systemic lupus erythematosus. Nat Commun. 2023; 14: 1388.
20. Zollars E, Bienkowska J, Czerkowicz J, et al. BAFF (B cell activating factor) transcript level in peripheral blood of patients with SLE is associated with same-day disease activity as well as global activity over the next year. Lupus Sci Med. 2015; 2: e000063.
21. Zollars E, Courtney S M, Wolf B J, et al. Clinical Application of a Modular Genomics Technique in Systemic Lupus Erythematosus: Progress towards Precision Medicine. Int J Genomics. 2016; 2016: 7862962.
22. Hochberg M C. Updating the American College of Rheumatology revised criteria for the classification of systemic lupus erythematosus. Arthritis and Rheumatism. 1997; 40: 1725-1725.
23. Petri M, Kim M Y, Kalunian K C, et al. Combined oral contraceptives in women with systemic lupus erythematosus. N. Engl. J. Med. 2005; 353: 2550-2558.
24. Petri M, Hellmann D. Hochberg M. Validity and reliability of lupus activity measures in the routine clinic setting. J. Rheumatol. 1992; 19: 53-59.
25. Gomez-Banuelos E, Goldman D W, Andrade V, et al. Uncoupling interferons and the interferon signature explains clinical and transcriptional subsets in SLE. Cell Rep Med. 2024; 101569.
26. Darrah E, Rosen A, Giles J T, Andrade F. Peptidylarginine deiminase 2, 3 and 4 have distinct specificities against cellular substrates: novel insights into autoantigen selection in rheumatoid arthritis. Ann. Rheum. Dis. 2012; 71: 92-98.
27. Banchereau R, Hong S, Cantarel B, et al. Personalized Immunomonitoring Uncovers Molecular Networks that Stratify Lupus Patients. Cell. 2016; 165: 551-565.
28. Huber W. Carey V J, Gentleman R, et al. Orchestrating high-throughput genomic analysis with Bioconductor. Nature Methods. 2015; 12: 115-121.
29. Li C, Xu J. Feature selection with the Fisher score followed by the Maximal Clique Centrality algorithm can accurately identify the hub genes of hepatocellular carcinoma. Sci Rep. 2019; 9: 17283.
30. You K S, Shung D. An R package for dimension reduction and intrinsic dimension estimation. Softw Impacts. 2022: 14:
31. Zhou Y, Zhou B, Pache L, et al. Metascape provides a biologist-oriented resource for the analysis of systems-level datasets. Nat Commun. 2019; 10: 1523.
32. Uesugi H, Ozaki S, Sobajima J, et al. Prevalence and characterization of novel PANCA, antibodies to the high mobility group non-histone chromosomal proteins HMG1 and HMG2, in systemic rheumatic diseases. J Rheumatol. 1998; 25: 703-709.
33. Reimer G, Rubin R L, Kotzin B L, Tan E M. Anti-native DNA antibodies from autoimmune sera also bind to DNA in mitochondria. J Immunol. 1984; 133: 2532-2536.
34. Galli M, Luciani D, Bertolini G, Barbui T. Lupus anticoagulants are stronger risk factors for thrombosis than anticardiolipin antibodies in the antiphospholipid syndrome: a systematic review of the literature. Blood. 2003; 101: 1827-1832.
35. Previtali S, Barbui T, Galli M. Anti-beta2-glycoprotein I and anti-prothrombin antibodies in antiphospholipid-negative patients with thrombosis: a case control study. Thromb Haemost. 2002; 88: 729-732.
36. Dieude M, Correa J A, Neville C, et al. Association of autoantibodies to heat-shock protein 60 with arterial vascular events in patients with antiphospholipid antibodies. Arthritis Rheum. 2011; 63: 2416-2424.
37. Petri M. Antiphospholipid syndrome. Transl Res. 2020; 225: 70-81.
38. Oke V. Gunnarsson I, Dorschner J, et al. High levels of circulating interferons type I, type II and type III associate with distinct clinical features of active systemic lupus erythematosus. Arthritis Res Ther. 2019; 21: 107.
39. Petri M, Rheinschmidt M, Whiting-O'Keefe Q, Hellmann D, Corash L. The frequency of lupus anticoagulant in systemic lupus erythematosus. A study of sixty consecutive patients by activated partial thromboplastin time, Russell viper venom time, and anticardiolipin antibody level. Ann Intern Med. 1987; 106: 524-531.
40. Wahl D G, Guillemin F, de Maistre E, et al. Risk for venous thrombosis related to antiphospholipid antibodies in systemic lupus erythematosus—a meta-analysis. Lupus. 1997; 6: 467-473.
41. Akhter E, Shums Z. Norman G L, et al. Utility of antiphosphatidylserine/prothrombin and IgA antiphospholipid assays in systemic lupus erythematosus. J Rheumatol. 2013; 40: 282-286.
42. Pengo V. Interaction between Antiphospholipid Antibodies and Protein C Anticoagulant Pathway: A Narrative Review. Semin Thromb Hemost. 2022; 48: 971-977.
43. Miao R, Jiang C, Chang W Y, et al. Gasdermin D permeabilization of mitochondrial inner and outer membranes accelerates and enhances pyroptosis. Immunity. 2023; 56: 2523-2541 e2528.
44. Tian C, Liu Y, Li Z, Zhu P, Zhao M. Mitochondria Related Cell Death Modalities and Disease. Front Cell Dev Biol. 2022; 10: 832356.
45. Lood C, Blanco L P, Purmalek M M, et al. Neutrophil extracellular traps enriched in oxidized mitochondrial DNA are interferogenic and contribute to lupus-like disease. Nat. Med. 2016; 22: 146-153.
46. Petretto A, Bruschi M, Pratesi F, et al. Neutrophil extracellular traps (NET) induced by different stimuli: A comparative proteomic analysis. PLoS One. 2019; 14: e0218946.
47. Bruschi M, Petretto A, Santucci L, et al. Neutrophil Extracellular Traps protein composition is specific for patients with Lupus nephritis and includes methyl-oxidized alphaenolase (methionine sulfoxide 93). Sci Rep. 2019; 9: 7934.
48. Caielli S, Cardenas J, de Jesus A A, et al. Erythroid mitochondrial retention triggers myeloid-dependent type I interferon in human SLE. Cell. 2021; 184: 4464-4479 e4419.
49. Melki I, Allaeys I, Tessandier N, et al. Platelets release mitochondrial antigens in systemic lupus erythematosus. Sci Transl Med. 2021; 13: eaav5928.
50. Melchinger H, Jain K, Tyagi T, Hwa J. Role of Platelet Mitochondria: Life in a Nucleus-Free Zone. Front Cardiovasc Med. 2019; 6: 153.

1. A method of identifying a subject suffering from systemic lupus erythematosus (SLE) that is at risk of thrombosis or malignancy, the method comprising the steps of:
a. contacting at least one biological sample obtained from a subject suffering from SLE with:
i. at least one first specific binding partner, wherein the at least one first specific binding partner comprises a polypeptide comprising amino acids 43 to 246 of mature human transcription factor A, mitochondrial (TFAM), and further wherein at least one anti-TFAM antibody in the sample specifically binds to the polypeptide, and
ii. at least one type of second specific binding partner comprising a detectable label, wherein the second specific binding partner specifically binds to at least one anti-TFAM antibody in the sample, thereby producing one or more types of first complexes comprising the first specific binding partner-anti-TFAM antibody-second specific binding partner;
b. assessing a signal from one or more types of first complexes, wherein the amount of detectable signal from the detectable label indicates the amount of at least one anti-TFAM antibody in the sample; and
c. identifying the subject as at risk of thrombosis or for malignancy if the amount of first complexes in the sample higher than a reference level or not at risk of thrombosis or for malignancy if the amount of first complexes in the sample is equal to or lower than a reference level, wherein the reference level is based on the amount of first complexes detected in a control group of human subjects who do not suffer from SLE.

2. The method of claim 1, wherein the human TEAM has at least 80%, at least 85%, at least 90%, at least 95%, at least 99% or at least 100% sequence identity to SEQ ID NO:1.

3. The method of claim 1, wherein the first specific binding partner binds to a first epitope on an anti-TFAM antibody and the second specific binding partner binds to an antigen binding site or paratope on the anti-TFAM antibody.

4. The method of claim 1, wherein the biological sample is a body fluid from a human subject.

5. The method of claim 4, wherein the body fluid is selected from the group consisting of whole blood, plasma, serum, salvia, ascites fluid and bronchoalveolar lavage.

6. The method of claim 1, wherein the at least one first specific binding partner or the at least one second specific binding partner is immobilized on a solid support.

7. The method of claim 1, wherein the second specific binding partner comprises a anti-human IgG, IgA, IgD, IgE or IgM antibody

8. The method of claim 1, wherein the reference level is at least one standard deviation above a mean amount of first complexes detected in a control group of human subjects who do not suffer from SLE.

9. The method of claim 8, wherein the reference level is at least two standard deviations above a mean amount of first complexes detected in a control group of human subjects who do not suffer from SLE.

10. The method of claim 1, wherein the method is selected from the group consisting of: an immunoassay, a clinical chemistry assay, and a lateral flow assay.

11. The method of claim 1, wherein the method is adapted for use in an automated system or a semi-automated system.

12. The method of claim 1, wherein the subject is identified as at risk of new or recurrent thrombotic events.

13. The method of claim 12, wherein the method comprises treating the subject identified as at risk of new or recurrent thrombotic events with at least one antithrombotic agents.

14. The method of claim 13, wherein the antithrombotic agents is low-dose aspirin, direct factor Xa inhibitors, thrombin inhibitors, PY12 inhibitors, low molecular weight inhibitors, warfarin, heparin, or a combination thereof.

15. The method of claim 1, wherein the subject is identified as at risk for malignancy.

16. The method of claim 15, wherein the subject is monitored for developing a malignancy.
